(19) United States
(12) Patent Application Publication
(10) Pub. No.: US 2018/0108431 A1
(43) Pub. Date: Apr. 19, 2018

(54) METHODS AND SYSTEMS FOR ASSESSING FERTILITY BASED ON SUBCLINICAL GENETIC FACTORS

(71) Applicant: Celmatix Inc., New York, NY (US)

(72) Inventors: Piraye Yurttas Beim, New York, NY (US); Emlyn Parfitt, New York, NY (US); Tina Hu-Seliger, New York, NY (US); Anthony Santistevan, New York, NY (US)

(21) Appl. No.: 15/728,442

(22) Filed: Oct. 9, 2017

Related U.S. Application Data
(60) Provisional application No. 62/408,632, filed on Oct. 14, 2016.

(51) Int. Cl. G16H 10/60; G01N 33/74; G01N 33/68

(52) U.S. Cl. CPC G16H 10/60; G01N 33/76; G01N 33/689; G01N 33/743

(57) ABSTRACT

The invention provides methods for generating a likelihood of achieving ongoing pregnancy in an individual by combining both clinical and genetic data. These methods involve the determination of one or more correlations between clinical characteristics and known pregnancy and infertility-related outcomes from a reference set of data to provide a model representative of a cumulative probability of ongoing pregnancy. The methods further involve the determination of one or more correlations between genetic characteristics and known pregnancy and infertility-related outcomes from the reference set of data to adjust the model. The model can then be applied to the input data to generate the likelihood of achieving ongoing pregnancy in the subject.

### CROSS-REFERENCE TO RELATED APPLICATIONS

This application claims the benefit of and priority to U.S. Provisional Application Ser. No. 62/408,632, file Oct. 14, 2016, the contents of which are incorporated herein by reference in their entirety.

### BACKGROUND

Approximately one in seven couples has difficulty conceiving. Infertility may be due to a single cause in either partner, or a combination of factors (e.g., genetic factors, diseases, or environmental factors) that may prevent a pregnancy from occurring or continuing.

From the time a couple seeks medical assistance for difficulty conceiving, the couple is advised to undergo a number of diagnostic procedures to ascertain potential causes for why the couple is having difficulty conceiving. Often the procedures can be highly invasive, costly, and time consuming. Furthermore, even after a couple has undergone these diagnostic procedures and has been informed as to their prognosis for achieving a live birth (LB), and subsequently makes treatment decisions based on this prognosis, the outcome may not be in line with the original prognosis.

The uncertainty surrounding prognoses for couples trying to conceive is a significant challenge for fertility specialists. This is especially the case when good prognosis patients fail treatment for unexplained reasons or when poor prognosis patients achieve live birth despite the odds.

### SUMMARY

The invention relates to methods and systems for assessing fertility and informing course of treatment. The invention provides methods for generating a likelihood of achieving pregnancy using a combination of clinical and genomic data. In one embodiment, the invention provides methods for assessing a cumulative probability of pregnancy over a number of in-vitro fertilization cycles. In a preferred embodiment, methods of the invention provide personalized data regarding the probability of achieving pregnancy based upon known clinical indicia overlayed by genomic classification data. Thus, clinical indicia of the probability of achieving pregnancy, such as age, BMI, and others provide an initial set of probabilities over N cycles of in vitro fertilization IVF. According to the invention, classification data (e.g., relating to oogenesis or ovarian reserve, genomic markers, etc) are applied to the clinical indicia in order to achieve a more precise probability of achieving pregnancy. In one aspect, the probability of achieving pregnancy is determined over the course of the N IVF cycles. FIG. 1 depicts typical results in which the stepwise curve marked BB is the probability curve based upon clinical indicia (e.g., phenotypic markers related to the likelihood of pregnancy), the CC curve is an exemplary displacement curve based upon negative genomic classification data, and the DD curve is an exemplary displacement curve based upon positive genomic classification data.

Methods of the invention provide advantages over previous studies that either looked at the genetics of particular reproductive conditions or were case-control studies that focused on allele frequencies in groups of patients defined by clinical diagnosis and/or prognosis. Methods of the present invention are not limited to discrete determinations and categorizations and, as such, provide more accurate, robust, and personalized models for assessing the likelihood of achieving ongoing pregnancy/live birth.

### BRIEF DESCRIPTION OF DRAWINGS

FIG. 1 depicts the cumulative probability of achieving ongoing pregnancy determined based upon clinical indicia and adjusted for the genomic classification data.

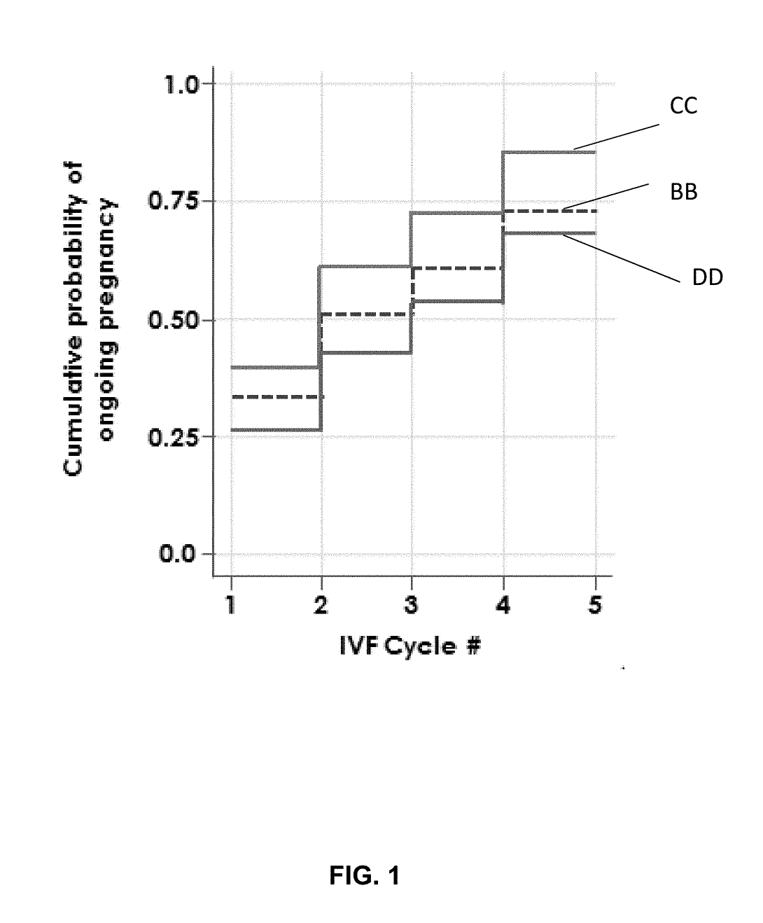

FIG. 2 depicts female reproduction/fertility related functional biological classifications.

FIG. 3 depicts male reproduction/fertility related functional biological classifications.

FIG. 4 depicts spermatogenic functional biological classifications.

FIG. 5 depicts a method for determining the impact of genetic characteristics on the cumulative probability of achieving ongoing pregnancy.

FIG. 6 depicts the cumulative probability of achieving ongoing pregnancy based on clinical characteristics of a reference set of data.

FIG. 7 depicts a general overview of the sequence kernel association test (SKAT) method for determining the effect of genetic characteristics on the cumulative probably of achieving ongoing pregnancy.

FIG. 8 depicts the cumulative probability of achieving ongoing pregnancy based on clinical characteristics of a reference set of data and adjusted for the SKAT-analysis results.

FIG. 9 depicts the cumulative probability of achieving ongoing pregnancy adjusted for the burden of deleterious mutations on various gene sets (genes or biological classifications).

FIG. 10 depicts a method for filtering through variants detected in whole genome sequencing for the identification of genetic regions related to infertility.

FIG. 11 depicts some of the components of the FertilomeTMDatabase, a tool for correlating genetic regions with risk for infertility (FertilomeTMScore).

FIG. 12 is a bioinformatics pipeline used to identify biologically interesting and statistically significant genetic variants in infertile patients.

FIG. 13 depicts a methodology for integrating clinical data with genomic data to predict treatment dependent and independent fertility outcomes.

FIG. 14 represents a diagram of a system of the invention.

### DETAILED DESCRIPTION

The invention relates to methods and systems for assessing likelihood of achieving pregnancy and/or live birth (LB) and for therapeutic intervention to achieve pregnancy. The invention provides methods for generating a likelihood of achieving ongoing pregnancy in an individual by combining both clinical and genetic data. These methods involve the determination of one or more correlations between clinical characteristics and known pregnancy and infertility-related outcomes from a reference set of data to provide a model representative of a cumulative probability of ongoing pregnancy. The methods further involve the determination of one or more correlations between genetic characteristics and known pregnancy and infertility-related outcomes from the reference set of data to adjust the model. The model can then be applied to the input data to generate the likelihood of achieving ongoing pregnancy in the subject.

### Genetic Data

In one aspect of the invention, genetic data includes genetic biomarkers and genetic classifications. These biomarkers and classifications can be utilized to provide more accurate prognoses that can inform downstream diagnostic tests and treatments that may benefit the subject.

Biomarkers for use with methods of the invention may be any marker that is associated with infertility/time to achieving ongoing pregnancy. Exemplary biomarkers include genes (e.g. any region of DNA encoding a functional product), genetic regions (e.g. regions including genes and intergenic regions with a particular focus on regions conserved throughout evolution in placental mammals), and gene products (e.g., RNA and protein). In certain embodiments, the biomarker is an infertility-associated gene or genetic region. An infertility-associated genetic region is any DNA sequence in which variation is associated with a change in fertility. Examples of changes in fertility include, but are not limited to, the following: a homozygous mutation of an infertility-associated gene leads to a complete loss of fertility; a homozygous mutation of an infertility-associated gene is incompletely penetrant and leads to reduction in fertility that varies from individual to individual; a heterozygous mutation is completely recessive, having no effect on fertility; and the infertility-associated gene is X-linked, such that a potential defect in fertility depends on whether a non-functional allele of the gene is located on an inactive X chromosome (Barr body) or on an expressed X chromosome.

In particular embodiments, the assessed infertility-associated genetic region is a maternal effect gene. Maternal effects genes are genes that have been found to encode key structures and functions in mammalian oocytes (Yurttas et al., Reproduction 139:809-823, 2010). Maternal effect genes are described, for example in, Christians et al. (Mol Cell Biol 17:778-88, 1997); Christians et al., Nature 407:693-694, 2000); Xiao et al. (EMBO J 18:5943-5952, 1999); Tong et al. (Endocrinology 145:1427-1434, 2004); Tong et al. (Nat Genet 26:267-268, 2000); Tong et al. (Endocrinology, 140:3720-3726, 1999); Tong et al. (Hum Reprod 17:903-911, 2002); Ohsugi et al. (Development 135:259-269, 2008); Borowczyk et al. (Proc Natl Acad Sci USA., 2009); and Wu (Hum Reprod 24:415-424, 2009). Maternal effects genes are also described in U.S. Ser. No. 12/889,304. The content of each of these is incorporated by reference herein in its entirety.

In particular embodiments, the infertility-associated genetic region is one or more genes (including exons, introns, and 10 kb of DNA flanking either side of said gene) selected from the genes shown in Table 1 below. In Table 1, OMIM reference numbers are provided when available.

TABLE 1
Human Infertility-Related Genes (OMIM #)
Table 1-Human Infertility-Related Genes (OMIM #)

| | | | |
|---|---|---|---|
| ABCA1 (600046) | ACTL6A (604958) | ACTL8 | ACVR1 (102576) |
| ACVR1B (601300) | ACVR1C (608981) | ACVR2 (102581) | ACVR2A (102581) |
| ACVR2B (602730) | ACVRL1 (601284) | ADA (608958) | ADAMTS1 (605174) |
| ADM (103275) | ADM2 (608682) | AFF2 (300806) | AGT (106150) |
| AHR (600253) | AIRE (607358) | AK2 (103020) | AK7 |
| AKR1C1 (600449) | AKR1C2 (600450) | AKR1C3 (603966) | AKR1C4 (600451) |
| AKT1 (164730) | ALDOA (103850) | ALDOB (612724) | ALDOC (103870) |
| ALPL (171760) | AMBP (176870) | AMD1 (180980) | AMH (600957) |
| AMHR2 (600956) | ANK3 (600465) | ANXA1 (151690) | APC (611731) |
| APOA1 (107680) | APOE (107741) | AQP4 (600308) | AR (313700) |
| AREG (104640) | ARF1 (103180) | ARF3 (103190) | ARF4 (601177) |
| ARF5 (103188) | ARFRP1 (604699) | ARL1 (603425) | ARL10 (612405) |
| ARL11 (609351) | ARL13A | ARL13B (608922) | ARL15 |
| ARL2 (601175) | ARL3 (604695) | ARL4A (604786) | ARL4C (604787) |
| ARL4D (600732) | ARL5A (608960) | ARL5B (608909) | ARL5C |
| ARL6 (608845) | ARL8A | ARL8B | ARMC2 |
| ARNTL (602550) | ASCL2 (601886) | ATF7IP (613644) | ATG7 (608760) |
| ATM (607585) | ATR (601215) | ATXN2 (601517) | AURKA (603072) |
| AURKB (604970) | AUTS2 (607270) | BARD1 (601593) | BAX (600040) |
| BBS1 (209901) | BBS10 (610148) | BBS12 (610683) | BBS2 (606151) |
| BBS4 (600374) | BBS5 (603650) | BBS7 (607590) | BBS9 (607968) |
| BCL2 (151430) | BCL2L1 (600039) | BCL2L10 (606910) | BDNF (113505) |
| BECN1 (604378) | BHMT (602888) | BLVRB (600941) | BMP15 (300247) |
| BMP2 (112261) | BMP3 (112263) | BMP4 (112262) | BMP5 (112265) |
| BMP6 (112266) | BMP7 (112267) | BMPR1A (601299) | BMPR1B (603248) |
| BMPR2 (600799) | BNC1 (601930) | BOP1 (610596) | BRCA1 (113705) |
| BRCA2 (600185) | BRIP1 (605882) | BRSK1 (609235) | BRWD1 |
| BSG (109480) | BTG4 (605673) | BUB1 (602452) | BUB1B (602860) |
| C2orf86 (613580) | C3 (120700) | C3orf56 | C6orf221 (611687) |
| CA1 (114800) | CARD8 (609051) | CARM1 (603934) | CASP1 (147678) |
| CASP2 (600639) | CASP5 (602665) | CASP6 (601532) | CASP8 (601763) |
| CBS (613381) | CBX1 (604511) | CBX2 (602770) | CBX5 (604478) |
| CCDC101 (613374) | CCDC28B (610162) | CCL13 (601391) | CCL14 (601392) |
| CCL4 (182284) | CCL5 (187011) | CCL8 (602283) | CCND1 (168461) |
| CCND2 (123833) | CCND3 (123834) | CCNH (601953) | CCS (603864) |
| CD19 (107265) | CD24 (600074) | CD55 (125240) | CD81 (186845) |
| CD9 (143030) | CDC42 (116952) | CDK4 (123829) | CDK6 (603368) |
| CDK7 (601955) | CDKN1B (600778) | CDKN1C (600856) | CDKN2A (600160) |
| CDX2 (600297) | CDX4 (300025) | CEACAM20 | CEBPA (116897) |
| CEBPB (189965) | CEBPD (116898) | CEBPE (600749) | CEBPG (138972) |
| CEBPZ (612828) | CELF1 (601074) | CELF4 (612679) | CENPB (117140) |
| CENPF (600236) | CENPI (300065) | CEP290 (610142) | CFC1 (605194) |
| CGA (118850) | CGB (118860) | CGB1 (608823) | CGB2 (608824) |
| CGB5 (608825) | CHD7 (608892) | CHST2 (603798) | CLDN3 (602910) |
| COIL (600272) | COL1A2 (120160) | COL4A3BP (604677) | COMT (116790) |
| COPE (606942) | COX2 (600262) | CP (117700) | CPEB1 (607342) |
| CRHR1 (122561) | CRYBB2 (123620) | CSF1 (120420) | CSF2 (138960) |
| CSTF1 (600369) | CSTF2 (600368) | CTCF (604167) | CTCFL (607022) |
| CTF2P | CTGF (121009) | CTH (607657) | CTNNB1 (116806) |
| CUL1 (603134) | CX3CL1 (601880) | CXCL10 (147310) | CXCL9 (601704) |
| CXorf67 | CYP11A1 (118485) | CYP11B1 (610613) | CYP11B2 (124080) |
| CYP17A1 (609300) | CYP19A1 (107910) | CYP1A1 (108330) | CYP27B1 (609506) |
| DAZ2 (400026) | DAZL (601486) | DCTPP1 | DDIT3 (126337) |
| DDX11 (601150) | DDX20 (606168) | DDX3X (300160) | DDX43 (606286) |
| DEPDC7 (612294) | DHFR (126060) | DHFRL1 | DIAPH2 (300108) |
| DICER1 (606241) | DKK1 (605189) | DLC1 (604258) | DLGAP5 |
| DMAP1 (605077) | DMC1 (602721) | DNAJB1 (604572) | DNMT1 (126375) |
| DNMT3B (602900) | DPPA3 (608408) | DPPA5 (611111) | DPYD (612779) |
| DTNBP1 (607145) | DYNLL1 (601562) | ECHS1 (602292) | EEF1A1 (130590) |
| EEF1A2 (602959) | EFNA1 (191164) | EFNA2 (602756) | EFNA3 (601381) |
| EFNA4 (601380) | EFNA5 (601535) | EFNB1 (300035) | EFNB2 (600527) |
| EFNB3 (602297) | EGR1 (128990) | EGR2 (129010) | EGR3 (602419) |
| EGR4 (128992) | EHMT1 (607001) | EHMT2 (604599) | EIF2B2 (606454) |
| EIF2B4 (606687) | EIF2B5 (603945) | EIF2C2 (606229) | EIF3C (603916) |
| EIF3CL (603916) | EPHA1 (179610) | EPHA10 (611123) | EPHA2 (176946) |
| EPHA3 (179611) | EPHA4 (602188) | EPHA5 (600004) | EPHA6 (600066) |
| EPHA7 (602190) | EPHA8 (176945) | EPHB1 (600600) | EPHB2 (600997) |
| EPHB3 (601839) | EPHB4 (600011) | EPHB6 (602757) | ERCC1 (126380) |
| ERCC2 (126340) | EREG (602061) | ESR1 (133430) | ESR2 (601663) |
| ESR2 (601663) | ESRRB (602167) | ETV5 (601600) | EZH2 (601573) |
| EZR (123900) | FANCC (613899) | FANCG (602956) | FANCL (608111) |
| FAR1 | FAR2 | FASLG (134638) | FBN1 (134797) |
| FBN2 (612570) | FBN3 (608529) | FBRS (608601) | FBRSL1 |
| FBXO10 (609092) | FBXO11 (607871) | FCRL3 (606510) | FDXR (103270) |
| FGF23 (605380) | FGF8 (600483) | FGFBP1 (607737) | FGFBP3 |
| FGFR1 (136350) | FHL2 (602633) | FIGLA (608697) | FILIP1L (612993) |
| FKBP4 (600611) | FMN2 (606373) | FMR1 (309550) | FOLR1 (136430) |
| FOLR2 (136425) | FOXE1 (602617) | FOXL2 (605597) | FOXN1 (600838) |
| FOXO3 (602681) | FOXP3 (300292) | FRZB (605083) | FSHB (136530) |
| FSHR (136435) | FST (136470) | GALT (606999) | GBPS (611467) |
| GCK (138079) | GDF1 (602880) | GDF3 (606522) | GDF9 (601918) |
| GGT1 (612346) | GJA1 (121014) | GJA10 (611924) | GJA3 (121015) |
| GJA4 (121012) | GJA5 (121013) | GJA8 (600897) | GJB1 (304040) |
| GJB2 (121011) | GJB3 (603324) | GJB4 (605425) | GJB6 (604418) |
| GJB7 (611921) | GJC1 (608655) | GJC2 (608803) | GJC3 (611925) |
| GJD2 (607058) | GJD3 (607425) | GJD4 (611922) | GNA13 (604406) |
| GNB2 (139390) | GNRH1 (152760) | GNRH2 (602352) | GNRHR (138850) |
| GPC3 (300037) | GPRC5A (604138) | GPRC5B (605948) | GREM2 (608832) |
| GRN (138945) | GSPT1 (139259) | GSTA1 (138359) | H19 (103280) |
| H1FOO (142709) | HABP2 (603924) | HADHA (600890) | HAND2 (602407) |
| HBA1 (141800) | HBA2 (141850) | HBB (141900) | HELLS (603946) |
| HK3 (142570) | HMOX1 (141250) | HNRNPK (600712) | HOXA11 (142958) |
| HPGD (601688) | HS6ST1 (604846) | HSD17B1 (109684) | HSD17B12 (609574) |
| HSD17B2 (109685) | HSD17B4 (601860) | HSD17B7 (606756) | HSD3B1 (109715) |
| HSF1 (140580) | HSF2BP (604554) | HSP90B1 (191175) | HSPG2 (142461) |
| HTATIP2 (605628) | ICAM1 (147840) | ICAM2 (146630) | ICAM3 (146631) |
| IDH1 (147700) | IFI30 (604664) | IFITM1 (604456) | IGF1 (147440) |
| IGF1R (147370) | IGF2 (147470) | IGF2BP1 (608288) | IGF2BP2 (608289) |
| IGF2BP3 (608259) | IGF2BP3 (608259) | IGF2R (147280) | IGFALS (601489) |
| IGFBP1 (146730) | IGFBP2 (146731) | IGFBP3 (146732) | IGFBP4 (146733) |
| IGFBP5 (146734) | IGFBP6 (146735) | IGFBP7 (602867) | IGFBPL1 (610413) |
| IL10 (124092) | IL11RA (600939) | IL12A (161560) | IL12B (161561) |
| IL13 (147683) | IL17A (603149) | IL17B (604627) | IL17C (604628) |
| IL17D (607587) | IL17F (606496) | IL1A (147760) | IL1B (147720) |
| IL23A (605580) | IL23R (607562) | IL4 (147780) | IL5 (147850) |
| IL5RA (147851) | IL6 (147620) | IL6ST (600694) | IL8 (146930) |
| ILK (602366) | INHA (147380) | INHBA (147290) | INHBB (147390) |
| IRF1 (147575) | ISG15 (147571) | ITGA11 (604789) | ITGA2 (192974) |
| ITGA3 (605025) | ITGA4 (192975) | ITGA7 (600536) | ITGA9 (603963) |
| ITGAV (193210) | ITGB1 (135630) | JAG1 (601920) | JAG2 (602570) |
| JARID2 (601594) | JMY (604279) | KAL1 (300836) | KDM1A (609132) |
| KDM1B (613081) | KDM3A (611512) | KDM4A (609764) | KDM5A (180202) |
| KDM5B (605393) | KHDC1 (611688) | KIAA0430 (614593) | KIF2C (604538) |
| KISS1 (603286) | KISS1R (604161) | KITLG (184745) | KL (604824) |
| KLF4 (602253) | KLF9 (602902) | KLHL7 (611119) | LAMC1 (150290) |
| LAMC2 (150292) | LAMP1 (153330) | LAMP2 (309060) | LAMP3 (605883) |
| LDB3 (605906) | LEP (164160) | LEPR (601007) | LFNG (602576) |
| LHB (152780) | LHCGR (152790) | LHX8 (604425) | LIF (159540) |
| LIFR (151443) | LIMS1 (602567) | LIMS2 (607908) | LIMS3 |
| LIMS3L | LIN28 (611043) | LIN28B (611044) | LMNA (150330) |
| LOC613037 | LOXL4 (607318) | LPP (600700) | LYRM1 (614709) |
| MAD1L1 (602686) | MAD2L1 (601467) | MAD2L1BP | MAF (177075) |
| MAP3K1 (600982) | MAP3K2 (609487) | MAPK1 (176948) | MAPK3 (601795) |
| MAPK8 (601158) | MAPK9 (602896) | MB21D1 (613973) | MBD1 (156535) |
| MBD2 (603547) | MBD3 (603573) | MBD4 (603574) | MCL1 (159552) |
| MCM8 (608187) | MDK (162096) | MDM2 (164785) | MDM4 (602704) |
| MECP2 (300005) | MED12 (300188) | MERTK (604705) | METTL3 (612472) |
| MGAT1 (160995) | MITF (156845) | MKKS (604896) | MKS1 (609883) |
| MLH1 (120436) | MLH3 (604395) | MOS (190060) | MPPED2 (600911) |
| MRS2 | MSH2 (609309) | MSH3 (600887) | MSH4 (602105) |
| MSH5 (603382) | MSH6 (600678) | MST1 (142408) | MSX1 (142983) |
| MSX2 (123101) | MTA2 (603947) | MTHFD1 (172460) | MTHFR (607093) |
| MTO1 (614667) | MTOR (601231) | MTRR (602568) | MUC4 (158372) |
| MVP (605088) | MX1 (147150) | MYC (190080) | NAB1 (600800) |
| NAB2 (602381) | NATI (108345) | NCAM1 (116930) | NCOA2 (601993) |
| NCOR1 (600849) | NCOR2 (600848) | NDP (300658) | NFE2L3 (604135) |
| NLRP1 (606636) | NLRP10 (609662) | NLRP11 (609664) | NLRP12 (609648) |
| NLRP13 (609660) | NLRP14 (609665) | NLRP2 (609364) | NLRP3 (606416) |
| NLRP4 (609645) | NLRP5 (609658) | NLRP6 (609650) | NLRP7 (609661) |
| NLRP8 (609659) | NLRP9 (609663) | NNMT (600008) | NOBOX (610934) |
| NODAL (601265) | NOG (602991) | NOS3 (163729) | NOTCH1 (190198) |
| NOTCH2 (600275) | NPM2 (608073) | NPR2 (108961) | NR2C2 (601426) |
| NR3C1 (138040) | NR5A1 (184757) | NR5A2 (604453) | NRIP1 (602490) |
| NRIP2 | NRIP3 (613125) | NTF4 (162662) | NTRK1 (191315) |
| NTRK2 (600456) | NUPR1 (614812) | OAS1 (164350) | OAT (613349) |
| OFD1 (300170) | OOEP (611689) | ORAI1 (610277) | OTC (300461) |
| PADI1 (607934) | PADI2 (607935) | PADI3 (606755) | PADI4 (605347) |
| PADI6 (610363) | PAEP (173310) | PAIP1 (605184) | PARP12 (612481) |
| PCNA (176740) | PCP4L1 | PDE3A (123805) | PDK1 (602524) |
| PGK1 (311800) | PGR (607311) | PGRMC1 (300435) | PGRMC2 (607735) |
| PIGA (311770) | PIM1 (164960) | PLA2G2A (172411) | PLA2G4C (603602) |
| PLA2G7 (601690) | PLAC1L | PLAG1 (603026) | PLAGL1 (603044) |
| PLCB1 (607120) | PMS1 (600258) | PMS2 (600259) | POF1B (300603) |
| POLG (174763) | POLR3A (614258) | POMZP3 (600587) | POU5F1 (164177) |
| PPID (601753) | PPP2CB (176916) | PRDM1 (603423) | PRDM9 (609760) |
| PRKCA (176960) | PRKCB (176970) | PRKCD (176977) | PRKCDBP |
| PRKCE (176975) | PRKCG (176980) | PRKCQ (600448) | PRKRA (603424) |
| PRLR (176761) | PRMT1 (602950) | PRMT10 (307150) | PRMT2 (601961) |
| PRMT3 (603190) | PRMT5 (604045) | PRMT6 (608274) | PRMT7 (610087) |
| PRMT8 (610086) | PROK1 (606233) | PROK2 (607002) | PROKR1 (607122) |
| PROKR2 (607123) | PSEN1 (104311) | PSEN2 (600759) | PTGDR (604687) |
| PTGER1 (176802) | PTGER2 (176804) | PTGER3 (176806) | PTGER4 (601586) |
| PTGES (605172) | PTGES2 (608152) | PTGES3 (607061) | PTGFR (600563) |
| PTGFRN (601204) | PTGS1 (176805) | PTGS2 (600262) | PTN (162095) |
| PTX3 (602492) | QDPR (612676) | RAD17 (603139) | RAX (601881) |
| RBP4 (180250) | RCOR1 (607675) | RCOR2 | RCOR3 |
| RDH11 (607849) | REC8 (608193) | REXO1 (609614) | REXO2 (607149) |
| RFPL4A (612601) | RGS2 (600861) | RGS3 (602189) | RSPO1 (609595) |
| RTEL1 (608833) | SAFB (602895) | SAR1A (607691) | SAR1B (607690) |
| SCARB1 (601040) | SDC3 (186357) | SELL (153240) | SEPHS1 (600902) |
| SEPHS2 (606218) | SERPINA10 (605271) | SFRP1 (604156) | SFRP2 (604157) |
| SFRP4 (606570) | SFRP5 (604158) | SGK1 (602958) | SGOL2 (612425) |
| SH2B1 (608937) | SH2B2 (605300) | SH2B3 (605093) | SIRT1 (604479) |
| SIRT2 (604480) | SIRT3 (604481) | SIRT4 (604482) | SIRT5 (604483) |
| SIRT6 (606211) | SIRT7 (606212) | SLC19A1 (600424) | SLC28A1 (606207) |
| SLC28A2 (606208) | SLC28A3 (608269) | SLC2A8 (605245) | SLC6A2 (163970) |
| SLC6A4 (182138) | SLCO2A1 (601460) | SLITRK4 (300562) | SMAD1 (601595) |
| SMAD2 (601366) | SMAD3 (603109) | SMAD4 (600993) | SMAD5 (603110) |
| SMAD6 (602931) | SMAD7 (602932) | SMAD9 (603295) | SMARCA4 (603254) |
| SMARCA5 (603375) | SMC1A (300040) | SMC1B (608685) | SMC3 (606062) |
| SMC4 (605575) | SMPD1 (607608) | SOCS1 (603597) | SOD1 (147450) |
| SOD2 (147460) | SOD3 (185490) | SOX17 (610928) | SOX3 (313430) |
| SPAG17 | SPARC (182120) | SPIN1 (609936) | SPN (182160) |
| SPO11 (605114) | SPP1 (166490) | SPSB2 (611658) | SPTB (182870) |
| SPTBN1 (182790) | SPTBN4 (606214) | SRCAP (611421) | SRD5A1 (184753) |
| SRSF4 (601940) | SRSF7 (600572) | ST5 (140750) | STAG3 (608489) |
| STAR (600617) | STARD10 | STARD13 (609866) | STARD3 (607048) |
| STARD3NL (611759) | STARD4 (607049) | STARD5 (607050) | STARD6 (607051) |
| STARD7 | STARD8 (300689) | STARD9 (614642) | STAT1 (600555) |
| STAT2 (600556) | STAT3 (102582) | STAT4 (600558) | STAT5A (601511) |
| STAT5B (604260) | STAT6 (601512) | STC1 (601185) | STIM1 (605921) |
| STK3 (605030) | SULT1E1 (600043) | SUZ12 (606245) | SYCE1 (611486) |
| SYCE2 (611487) | SYCP1 (602162) | SYCP2 (604105) | SYCP3 (604759) |
| SYNE1 (608441) | SYNE2 (608442) | TAC3 (162330) | TACC3 (605303) |
| TACR3 (162332) | TAF10 (600475) | TAF3 (606576) | TAF4 (601796) |
| TAF4B (601689) | TAF5 (601787) | TAF5L | TAF8 (609514) |
| TAF9 (600822) | TAP1 (170260) | TBL1X (300196) | TBXA2R (188070) |
| TCL1A (186960) | TCL1B (603769) | TCL6 (604412) | TCN2 (613441) |
| TDGF1 (187395) | TERC (602322) | TERF1 (600951) | TERT (187270) |
| TEX12 (605791) | TEX9 | TF (190000) | TFAP2C (601602) |
| TFPI (152310) | TFPI2 (600033) | TG (188450) | TGFB1 (190180) |
| TGFB1I1 (602353) | TGFBR3 (600742) | THOC5 (612733) | THSD7B |
| TLE6 (612399) | TM4SF1 (191155) | TMEM67 (609884) | TNF (191160) |
| TNFAIP6 (600410) | TNFSF13B (603969) | TOP2A (126430) | TOP2B (126431) |
| TP53 (191170) | TP53I3 (605171) | TP63 (603273) | TP73 (601990) |
| TPMT (187680) | TPRXL (611167) | TPT1 (600763) | TRIM32 (602290) |
| TSC2 (191092) | TSHB (188540) | TSIX (300181) | TTC8 (608132) |
| TUBB4Q (158900) | TUFM (602389) | TYMS (188350) | UBB (191339) |
| UBC (191340) | UBD (606050) | UBE2D3 (602963) | UBE3A (601623) |
| UBL4A (312070) | UBL4B (611127) | UIMC1 (609433) | UQCR11 (609711) |
| UQCRC2 (191329) | USP9X (300072) | VDR (601769) | VEGFA (192240) |
| VEGFB (601398) | VEGFC (601528) | VHL (608537) | VIM (193060) |
| VKORC1 (608547) | VKORC1L1 (608838) | WAS (300392) | WISP2 (603399) |
| WNT7A (601570) | WNT7B (601967) | WT1 (607102) | XDH (607633) |
| XIST (314670) | YBX1 (154030) | YBX2 (611447) | ZAR1 (607520) |
| ZFX (314980) | ZNF22 (194529) | ZNF267 (604752) | ZNF689 |
| ZNF720 | ZNF787 | ZNF84 | ZP1 (195000) |
| ZP2 (182888) | ZP3 (182889) | ZP4 (613514) | |

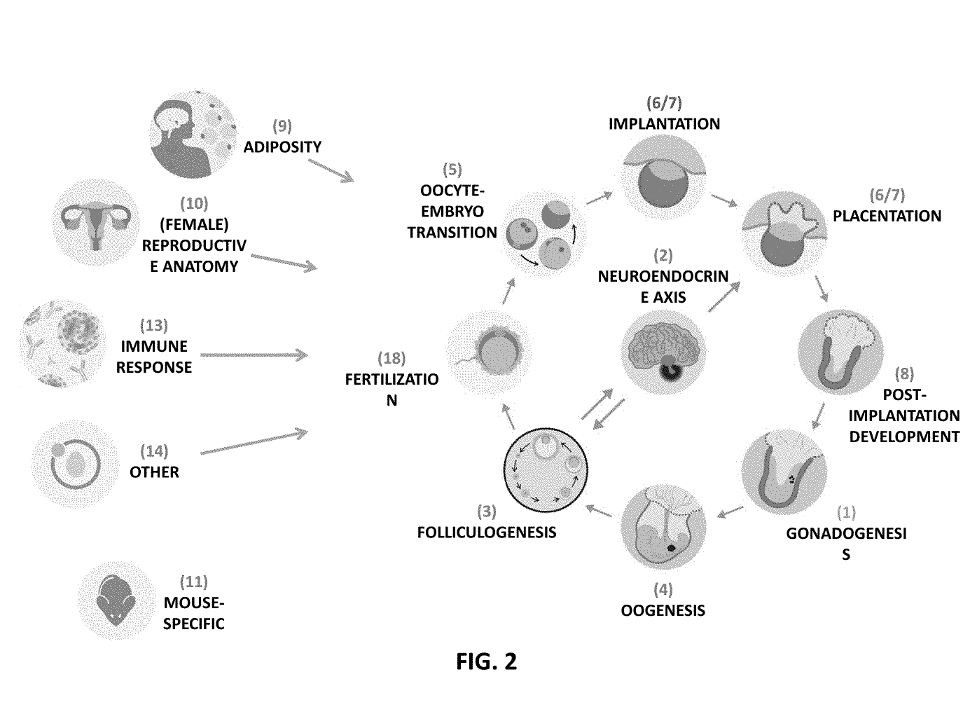

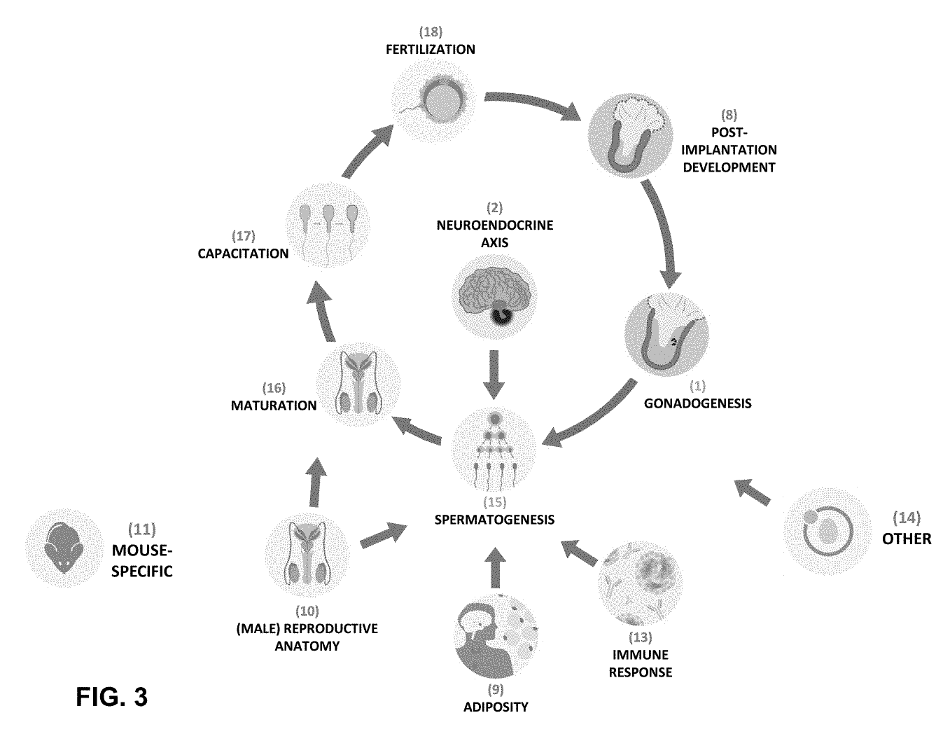

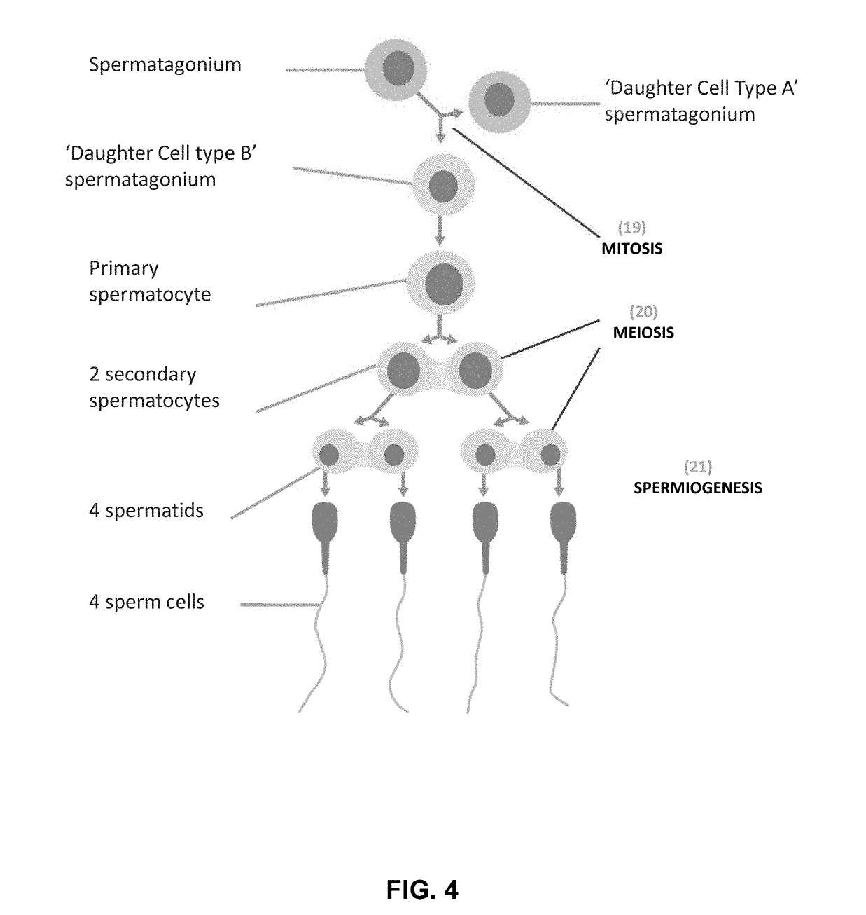

The genes listed in Table 1 can be involved in different aspects of reproduction/fertility related processes. Furthermore additional genes beyond those maternal effect genes listed in Table 1 can also affect fertility. Genes affecting fertility can be involved with a number of male- and female-specific processes, or functional biological classifications, such as those shown in FIGS. 2-4. As shown in FIG. 2, female reproductive/fertility related processes, or classifications, include gonadogenesis, neuroendocrine axis, folliculogensis, oogenesis, oocyte-embyro transition, placentation, post-implantation development, adiposity, (female) reproductive anatomy, immune response, fertilization and other processes. Male reproductive/fertility related processes, or classifications, include gonadogenesis neuroendocrine axis, post-implantation development, adiposity, (male) reproductive anatomy, immune response, spermatogenesis, sperm maturation and capacitation, fertilization, mitosis, meiosis, spermiogenesis, and other processes, as shown in FIGS. 3 and 4. These processes are described in more detail below.

Gonadogenesis encompasses the processes regulating the development of the ovaries and testes, and involves, but is not limited to, primordial germ cell specification and proliferation. The neuroendocrine axis encompasses for example the physiological pathways and structures regulating the production and activity of hormones in a number of different tissues in the human body, including the brain and gonads. Folliculogenesis encompasses the physiological mechanisms regulating the development of primordial follicles to cystic follicles in the ovary. Oogenesis encompasses the physiological mechanisms regulating the development of primordial oocytes to mature meiosis-II stage oocytes ready to be fertilized, hence those that are specific to female reproductive biology. Oocyte-embryo transition encompasses the physiological mechanisms regulating the development of the early embryo and includes mechanisms related to egg quality, such as oocyte cytoplasmic lattice formation, and paternal effect mechanisms. Placentation (Embryonic) encompasses the embryo-specific physiological mechanisms regulating implantation and the development of the placenta. Placentation (Uterine) encompasses the uterus-specific physiological mechanisms regulating embryo implantation and the development of the placenta. Post-implantation development encompasses the physiological mechanisms regulating post-implantation embryo development, particularly those whose disruption might lead to abnormal development or pregnancy loss in humans. Adiposity encompasses the physiological mechanisms regulating adipose tissue and body weight, which are known to play an important, indirect role in mammalian fecundity and infertility. Reproductive anatomy encompasses any phenotype relating to anatomical changes that could impact reproduction, fecundity or fertility. Immune response encompasses phenotypes that are specific to aspects of immune response mechanisms, which are known to play an important role in mammalian reproduction and fertility.

Spermatogenesis encompasses the processes involved in the production or development of mature spermatozoa, hence those that are specific to male reproductive biology. Maturation encompasses processes that enable spermatozoa to fertilize eggs, hence those that are specific to male reproductive biology. Capacitation encompasses processes specific to functional capacitation of spermatozoa in the vaginal canal and uterus. Fertilization encompasses processes relating to the union of a human egg and sperm. Mitosis encompasses processes involving changes to the cell division process such that it does not end with two daughter cells that have the same chromosomal complement as the parent cell. Such changes to the mitotic process may affect for example fertility-related cell proliferation or tissue maintenance. Meiosis encompasses processes regulating meiosis such that it results in four daughter cells each with exactly half the chromosome complement of the parent cell, for example during gametogenesis. Spermiogenesis encompasses processes regulating the morphological differentiation of haploid cells into sperm.

Table 2 lists examples of genes associated with various biological classifications, i.e. gene sets. Genes can be classified in other ways as well. For example, they can be sub-classified according to the cellular function they perform i.e. transcription factor, signaling molecule, ligand, receptor, cytoskeletal component. Alternatively, they could be classified according to the role they play on a tissue level e.g. proliferation, differentiation, apoptosis. As can be seen in Table 2, a gene can be associated with more than one biological classification. The gene sets are determined using a bioinformatics pipeline and associated databases, as described in more detail below.

TABLE 2

| Biological | |
|---|---|
| Classification | Genes |
| Gonadogenesis | BAX, BMP4, DAZL, DICER1, FMR1, NOG, NR5A1, PRDM1, |
| | SOX17, XPNPEP2 |
| Neuroendocrine | ACVR1, ACVR1B, AHR, AR, BRCA2, CDKN1B, CDKN1C, CENPI, |
| axis | CGB1, DAZL, DDX20, ESR1, ESR2, FOXE1, FOXL2, FSHR, FST, |
| | HAND2, HS6ST1, HSD17B1, HSD17B12, HSD17B2, HSD17B7, |
| | IGF1, INHA, KL, KLF4, LHB, LHCGR, MTA2, MTOR, NODAL, |
| | NR3C1, NR5A1, PLA2G4C, PRKRA, PRLR, SCARB1, SDC3, |
| | TAF4B, TGFB1, TSC2 |
| Folliculogenesis | ACVR1, ACVR1C, ACVR1C, AHR, AR, BAX, BMP15, BMP4, |
| | BMP7, CDKN1B, CENPI, DDX20, EEF1A1, EIF2B2, EIF2B5, |
| | ESR1, ESR2, FRM1, FOXE1, FOXL2, FOXO3, FSHR, FST, |
| | GALT, GDF3, GDF9, IGF1, IL6ST, INHA, KLF4, LHB, LCGR, |
| | MCM8, MTOR, MYC, NOBOX, NOG, NTF4, OAS1, PRLR, |
| | PROKR1, PROKR2, TAF4B, TGFB1, TP73, TSC2, USP9X, |
| | WT1, XPNPEP2, ZFX, ZP2, ZP3 |
| Oogensis | ACTL6A, AHR, ATM, ATR, AURKA, AURKB, BARD1, BAX, |
| | BHMT, BMP15, BMP4, BMP7, BNC1, BRCA1, BRCA2, BUB1, |
| | CDK1B, CTCF, DAZL, DDX20, DIAPH2, EEF1A1, EIF2B2, |
| | EIF2B5, ESR2, FMN2, FMR1, FOXL2, FOXO3, GDF9, HSF1, |
| | IL6ST, KDM1B, KHDC1, KHDC3L, LHCGR, LIFR, MAD1L1, |
| | MAD2L1, MCM8, MTA2, MTOR, MTRR, MYC, NLRP11, |
| | NLRP13, NLRP14, NLRP4, NLRP5, NLRP7, NLRP8, NLRP9, |
| | NOBOX, NOG, NPM2, NTF4, OAS1, OOEP, PLA2G4C, PMS2, |
| | POLG, PRDM1, PRLR, RFPL4A, SCARB1 TACC3, TAF4B, |
| | TLE6, TP63, TP73 TSC2, ZFX, ZP1, ZP2, ZP3, ZP4 |
| Oocyte-embryo | ACTL6A, ATR, BARD1, BHMT, BNC1, BRCA1, BUB1, CD55, |
| transition | CENPF, CTCF, DAZL, DDX20, DNMT1, ESRRB, EZH2, EZR, |
| | HSD17B12, HSF1, IGF1, KDM1B, KHDC1, KHDC3L, LIFR, |
| | MTA2, MTOR, MYC, NLRP11, NLRP13, NLRP14, NLRP4, |
| | NLRP5, NLRP7, NLRP8, NLRP9, NPM2, OAS1, OOEP, PLA2G4C, |
| | PMS2, PRLR, RFPL4A, SMARCA4, SUZ12, TAF4B, TGFB1, |
| | TLE6, TP53, TP73, WT1, ZAR1, ZP1, ZP2, ZP3, ZP4 |
| Placentation | ACVR1B, ACVR1C, ASCL2, BMP4, BOP1, CD55, CDX4, ESRRB, |
| (Embryonic) | LIFR, NLRP7, PRDM1, SMARCA4, STK3, TDGF1 |
| Placentation | ACVR1, AR, ASCL2, BOP1, CDKN1C, CGB1, CGB2, DNMT1, |
| (Uterine) | EEF1A1, ESR1, EZR, FOLR2, GNA13, HADHA, HAND2, |
| | HS6ST1, HSF1, IL11RA, IL6ST, LIFR, MDM4, MST1, MTHFR, |
| | MUC4, MYC, NODAL, PRLR, PROK1, PROKR2, SDC3, SOCS1, |
| | TF, TFPI2, TGFB1, TP53, TSC2, WT1 |
| Post-Implantation | ACVR1B, ACVR1C, ATR, BARD1, BHMT, BMP4, BOP1, BRCA1, |
| Development | BUB1, CDX4, EZH2, GDF1, GDF3, GPC3, HSD17B12, KDM1B, |
| | MTOR, MYC, NODAL, SOX17, STK3, SUZ12, TACC3, TDGF1, |
| | TP53, TP63 |

Mutations in genes associated with these various processes result in fertility difficulties for males and/or females containing these mutations.

### Obtaining Genetic Data

Genetic data can be obtained, for example, by conducting an assay on a sample from a male or female that detects either a variant in an infertility-associated genetic region or abnormal (over or under) expression of an infertility-associated genetic region. The presence of certain variants in those genetic regions or abnormal expression levels of those genetic regions is indicative a fertility outcomes, i.e., whether ongoing pregnancy or live birth is achievable. Exemplary variants include, but are not limited to, a single nucleotide polymorphism, a single nucleotide variant, a deletion, an insertion, an inversion, a genetic rearrangement, a copy number variation, chromosomal microdeletion, genetic mosaicism, karyotype abnormality or a combination thereof.

A sample may include a human tissue or bodily fluid and may be collected in any clinically acceptable manner. A tissue is a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CSF. A sample may also be a fine needle aspirate or biopsied tissue, e.g. an endometrial aspirate, breast tissue biopsy, and the like. A sample also may be media containing cells or biological material. A sample may also be a blood clot, for example, a blood clot that has been obtained from whole blood after the serum has been removed. In certain embodiments, the sample may include reproductive cells or tissues, such as gametic cells, gonadal tissue, fertilized embryos, and placenta. In certain embodiments, the sample is blood, saliva, or semen collected from the subject.

Genetic information from the sample can be obtained by nucleic acid extraction from the sample. Methods for extracting nucleic acid from a sample are known in the art. See for example, Maniatis, et al., Molecular Cloning: A Laboratory Manual, Cold Spring Harbor, N.Y., pp. 280-281, 1982, the contents of which are incorporated by reference herein in their entirety. In certain embodiments, a sample is collected from a subject followed by enrichment for genes or gene fragments of interest, for example by hybridization to a nucleotide array including fertility-related genetic regions or genetic fragments of interest. The sample may be enriched for genetic regions of interest (e.g., infertility-associated genetic regions) using methods known in the art, such as hybrid capture. See for examples, Lapidus (U.S. Pat. No. 7,666,593), the content of which is incorporated by reference herein in its entirety.

In particular embodiments, the assay is conducted on fertility-related genes or genetic regions containing the gene or a part thereof, such as those genes found in Tables 1 and/or 2. Detailed descriptions of conventional methods, such as those employed to make and use nucleic acid arrays, amplification primers, hybridization probes, and the like can be found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, Calif.), Applied Biosystems (Foster City, Calif.), and Agilent Technologies (Santa Clara, Calif.).

Methods of detecting variations (e.g., mutations) are known in the art. In certain embodiments, a known single nucleotide polymorphism at a particular position can be detected by single base extension for a primer that binds to the sample DNA adjacent to that position. See for example Shuber et al. (U.S. Pat. No. 6,566,101), the content of which is incorporated by reference herein in its entirety. In other embodiments, a hybridization probe might be employed that overlaps the SNP of interest and selectively hybridizes to sample nucleic acids containing a particular nucleotide at that position. See for example Shuber et al. (U.S. Pat. Nos. 6,214,558 and 6,300,077), the content of which is incorporated by reference herein in its entirety.

In particular embodiments, nucleic acids are sequenced in order to detect variants in the nucleic acid compared to wild-type and/or non-mutated forms of the sequence. The nucleic acid can include a plurality of nucleic acids derived from a plurality of genetic elements. Methods of detecting sequence variants are known in the art, and sequence variants can be detected by any sequencing method known in the art.

DNA sequencing techniques include classic dideoxy sequencing reactions (Sanger method) using labeled terminators or primers and gel separation in slab or capillary, sequencing by synthesis using reversibly terminated labeled nucleotides, pyrosequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes

One conventional method to perform sequencing is by chain termination and gel separation, as described by Sanger et al., Proc Natl. Acad. Sci. USA, 74(12): 5463 67 (1977). Another conventional sequencing method involves chemical degradation of nucleic acid fragments. See, Maxam et al., Proc. Natl. Acad. Sci., 74: 560 564 (1977). Finally, methods have been developed based upon sequencing by hybridization. See, e.g., Harris et al., (U.S. patent application number 2009/0156412). The content of each reference is incorporated by reference herein in its entirety.

A sequencing technique that can be used in the methods of the provided invention includes, for example, Helicos True Single Molecule Sequencing (tSMS) (Harris T. D. et al. (2008) Science 320:106-109), incorporated herein by reference; see also, e.g., Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety. Another example of a DNA sequencing technique that can be used in the methods of the provided invention is 454 sequencing (Roche) (Margulies, M et al. 2005, Nature, 437, 376-380).

Another example of a DNA sequencing technique that can be used in the methods of the provided invention is SOLiD technology (Applied Biosystems). Another example of a DNA sequencing technique that can be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety.

Another example of a sequencing technology that can be used in the methods of the provided invention is next-gen sequencing, such as Illumina sequencing, using Illumina HiSeq sequencers. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni G V and Meller A. (2007) Clin Chem 53: 1996-2001, incorporated herein by reference). Another example of a sequencing technique that can be used in the methods of the provided invention involves using a chemical-sensitive field effect transistor (chemFET) array to sequence DNA (for example, as described in US Patent Application Publication No. 20090026082 and incorporated by reference). Another example of a sequencing technique that can be used in the methods of the provided invention involves using an electron microscope (Moudrianakis E. N. and Beer M. Proc Natl Acad Sci USA. 1965 March; 53:564-71, incorporated herein by reference).

In certain aspects, the invention provides a microarray including a plurality of oligonucleotides attached to a substrate at discrete addressable positions, in which at least one of the oligonucleotides hybridizes to a portion of a gene suspected of affecting fertility in a man or woman. Methods of constructing microarrays are known in the art. See for example Yeatman et al. (U.S. patent application number 2006/0195269), the content of which is hereby incorporated by reference in its entirety.

If the nucleic acid from the sample is degraded or only a minimal amount of nucleic acid can be obtained from the sample, PCR can be performed on the nucleic acid in order to obtain a sufficient amount of nucleic acid for sequencing (See e.g., Mullis et al. U.S. Pat. No. 4,683,195, the contents of which are incorporated by reference herein in its entirety).

Sequencing by any of the methods described above and known in the art produces sequence reads. Sequence reads can be analyzed to call variants by any number of methods known in the art. Variant calling can include aligning sequence reads to a reference (e.g. hg18) and reporting single nucleotide (SNP) alleles. An example of methods for analyzing sequence reads and calling variants includes standard Genome Analysis Toolkit (GATK) methods. See The Genome Analysis Toolkit: a MapReduce framework for analyzing next-generation DNA sequencing data, Genome Res 20(9):1297-1303, the contents of each of which are incorporated by reference. GATK is a software package for analysis of high-throughput sequencing data capable of identifying variants, including SNPs.

SNP alleles can be reported in a format such as a Sequence Alignment Map (SAM) or a Variant Call Format (VCF) file. Some background may be found in Li & Durbin, 2009, Fast and accurate short read alignment with Burrows-Wheeler Transform. Bioinformatics 25:1754-60 and McKenna et al., 2010. Variant calling produces results (“variant calls”) that may be stored as a sequence alignment map (SAM) or binary alignment map (BAM) file—comprising an alignment string (the SAM format is described, e.g., in Li, et al., The Sequence Alignment/Map format and SAMtools, Bioinformatics, 2009, 25(16):2078-9). Additionally or alternatively, output from the variant calling may be provided in a variant call format (VCF) file, e.g., in report. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described in Danecek et al., 2011, The variant call format and VCFtools, Bioinformatics 27(15):2156-2158. Further discussion may be found in U.S. Pub. 2013/0073214; U.S. Pub. 2013/0345066; U.S. Pub. 2013/0311106; U.S. Pub. 2013/0059740; U.S. Pub. 2012/0157322; U.S. Pub. 2015/0057946 and U.S. Pub. 2015/0056613, each incorporated by reference.

Furthermore, methods of the invention include conducting an assay on a sample from a subject that detects an abnormal (over or under) expression of an infertility-associated gene (e.g. a differentially or abnormally expressed gene). A differentially or abnormally expressed gene refers to a gene whose expression is activated to a higher or lower level in a subject suffering from a disorder, such as infertility, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, such as infertility, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

Methods of detecting levels of gene products (e.g., RNA or protein) are known in the art. Commonly used methods known in the art for the quantification of mRNA expression in a sample include northern blotting and in situ hybridization (Parker & Barnes, Methods in Molecular Biology 106:247 283 (1999), the contents of which are incorporated by reference herein in their entirety); RNAse protection assays (Hod, Biotechniques 13:852 854 (1992), the contents of which are incorporated by reference herein in their entirety); and PCR-based methods, such as reverse transcription polymerase chain reaction (RT-PCR) (Weis et al., Trends in Genetics 8:263 264 (1992), the contents of which are incorporated by reference herein in their entirety). Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure. Various methods are well known in the art. See, e.g., Ausubel et al., Current Protocols of Molecular Biology, John Wiley and Sons (1997); Rupp and Locker, Lab Invest. 56:A67 (1987), and De Andres et al., BioTechniques 18:42044 (1995); Held et al., Genome Research 6:986 994 (1996), the contents of which are incorporated by reference herein in their entirety.

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In another embodiment, a MassARRAY-based gene expression profiling method is used to measure gene expression. For further details see, e.g. Ding and Cantor, Proc. Natl. Acad. Sci. USA 100:3059 3064 (2003), incorporated herein by reference.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269), the content of which is incorporated by reference herein in its entirety. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996), the contents of which are incorporated by reference herein in their entirety). Microarray analysis can be performed by commercially available equipment, following manufacturer's protocols, such as by using the Affymetrix GenChip technology, or Incyte's microarray technology.

In another aspect, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes).

In yet another aspect, levels of transcripts of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. For more details see, e.g. Velculescu et al., Science 270:484 487 (1995); and Velculescu et al., Cell 88:243 51 (1997, the contents of each of which are incorporated by reference herein in their entirety).

In other embodiments, Massively Parallel Signature Sequencing (MPSS) is used to measure gene expression. For more details see, e.g. Brenner et al., Nature Biotechnology 18:630 634 (2000).

Immunohistochemistry methods are also suitable for detecting the expression levels of the gene products of the present invention. In these methods, antibodies (monoclonal or polyclonal) or antisera, such as polyclonal antisera, specific for each marker are used to detect expression. Immunohistochemistry protocols and kits are well known in the art and are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See, for example, U.S. Pat. Nos. 6,925,389; 6,989,100; and 6,890,763, each of which is incorporated by reference herein in their entirety.

### Defining Gene Sets

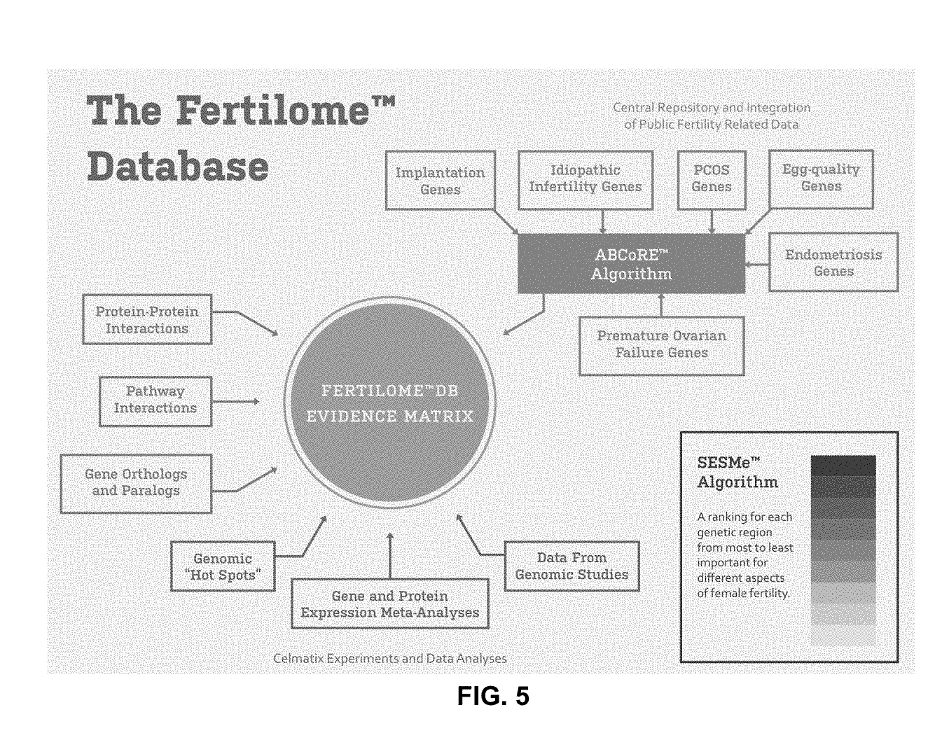

In accordance with methods of the present invention, gene sets, like the ones listed in Table 2, are used in models for assessing the cumulative probability of achieving ongoing pregnancy, as described in more detail below. Gene sets are defined using an infertility database (the Fertilome Database) comprised of various data sources, as illustrated in FIG. 5.

As shown in FIG. 5, information contained in the database is obtained from private and public fertility-related data. Private and/or public fertility-related data may include implantation genes, idiopathic infertility genes, polycystic ovary syndrome (PCOS) genes, egg quality genes, endometriosis genes, and premature ovarian failure genes. Although not shown here, the data may also include those genes involved in male and female functional biological classifications. The private and/or public fertility-related data is then subjected to an algorithm to provide genomic regions and variations of interest that can be introduced into a fertility database evidence matrix along with other fertility-related information.

In one embodiment, an algorithm identifying fertility regions of interest by performing evolutionary conservation analysis of one or more genes obtained from the private and/or public fertility-related data (the ABCoRE Algorithm) can be used. The other fertility-related information includes, for example, protein-protein interactions, pathway interactions, gene orthologs and paralogs, genomic “hotpsots”, gene protein expression and meta-analysis, and data from genomic studies. In operation, whole genomic sequencing data is compared to the compiled data in the fertility database evidence matrix to facilitate identification of potential genetic regions important for fertility. The fertility database evidence matrix filters through WGS variants to identify variants of fertility significance.

In certain embodiments, the whole genomic sequencing data can be subjected to an algorithm that ranks each genetic region from most to least important for different aspects of male and female fertility. In one example, as also shown in FIG. 5, an algorithm is used to rank each genetic region from most to least important for different aspects of female fertility (the SESMe algorithm), but can be expanded to include different aspects of male fertility as well. Any number of ranking schemes known in the art and/or one or more of the ranking schemes described in more detail in co-owned U.S. patent application Ser. No. 14/605,452, the contents of which are incorporated herein in its entirety, can be used.

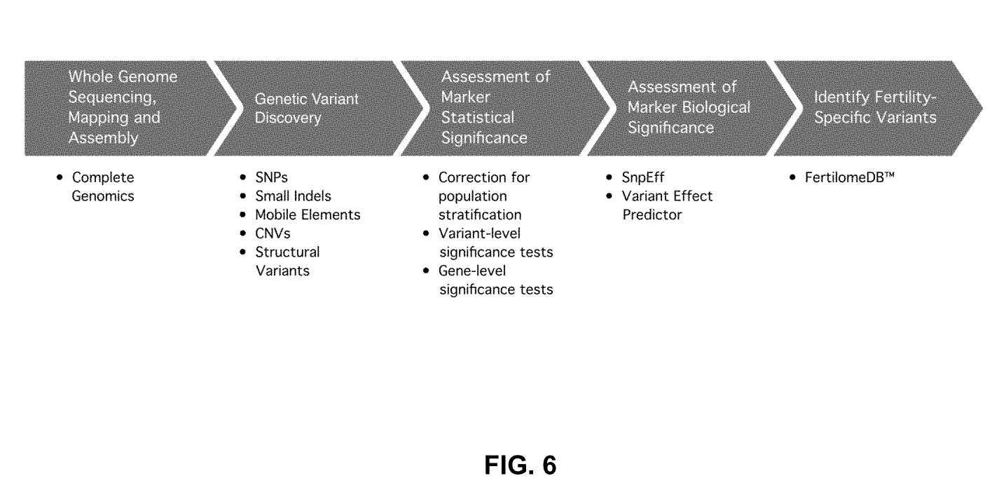

FIG. 6 illustrates a bioinformatics pipeline used to filter through WGS data to identify biomarkers associated with infertility according to certain embodiments, the data of which are eventually used as inputs to the infertility-associated database (the Fertilome Database) shown in FIG. 5. Whole genome sequencing (WGS) allows one to characterize the complete nucleic acid sequence of an individual's genome. With the amount of data obtained from WGS, a comprehensive collection of an individual's genetic variation is obtainable, which provides great potential for genetic biomarker discovery. The data obtained from WGS can be advantageously used to expand the ability to identify and characterize male and female infertility biomarkers. However, the ability to identify unknown variations of fertility significance within the vast WGS datasets is a challenging task that is analogous to finding a needle in a haystack. As shown in FIG. 6, samples are subjected to whole genome sequencing, mapping, and assembly. The WGS data is then analyzed to discover genetic variants such as SNPs, small indels, mobile elements, copy number variations, and structural variations. The identified variations are then assessed for statistical significance. This includes correction for population stratification, variation-level significance tests, and gene level significance tests. In addition, the biological significance of WGS variants is determined using, for example, the SnpEff and Variant Effect Predictor (www.ensembl.org) engines. SnpEff is capable of rapidly categorizing the effects of SNPs and other variants in whole genome sequences. See, Cingolani et al., A program for annotating and predicting the effects of single nucleotide polymorphisms, SnpEff: SNPs in the genome of Drosophila melanogaster strain w¹¹¹⁸; iso-2; iso-3; Landes Bioscience, 6:2, 1-13; April/May/June 2012, incorporated herein by reference. Variants of biological and statistical significance are then entered into the infertility knowledgebase in order to classify those variants as fertility biomarkers and define gene sets.

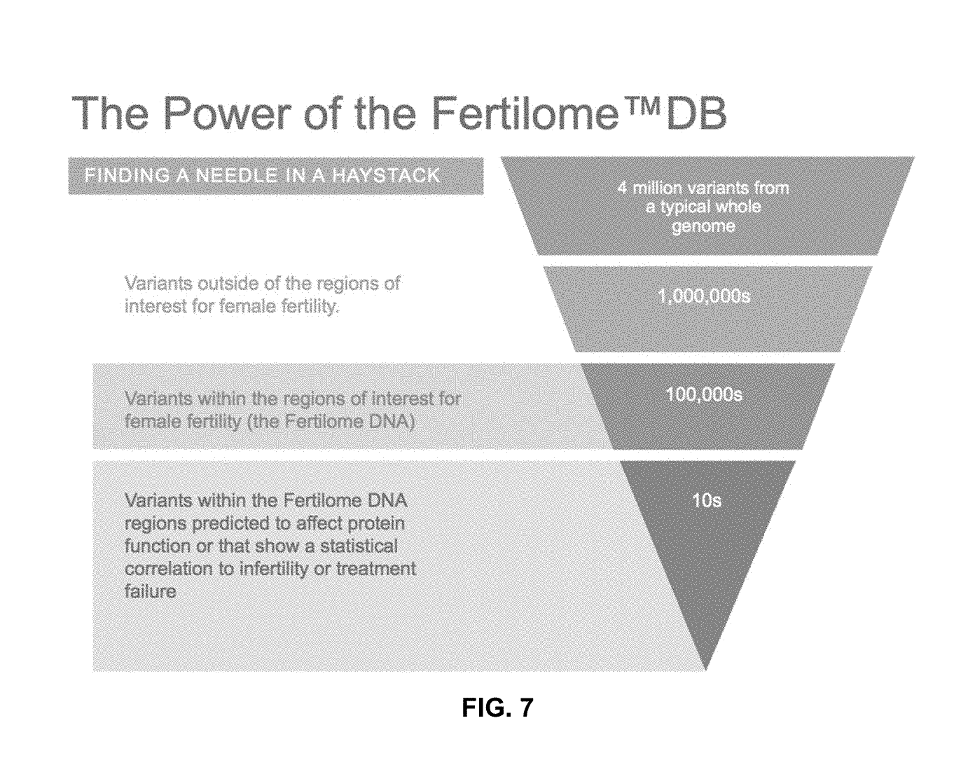

FIG. 7 generally illustrates the power of using an infertility knowledgebase to filter through variations obtained from WGS sequencing data in order to identify variations of infertility significance. As shown in FIG. 7, a typical whole genome can include up to four million variants. In accordance with method of the invention, variants outside of regions of interest for female fertility (which amounts to about one million variants) are first filtered out. Next, the filtering method isolates variants within regions of interest for female fertility. In one embodiment, regions of the human genome that control egg quality and fertility can be described as Fertilome nucleic acid. Variations located within the Fertilome nucleic acid may be in the 100,000s. The variations within the Fertilome nucleic acid can be filtered further to identify and score variations of infertility significance. Particularly, variations of infertility significance include those within regions predicted to effect biological function or that show a statistical correlation to infertility or treatment failure. It is to be understood that the illustrated method can be expanded and/or modified to include regions of interest for male fertility and/or combined male and female fertility.

### Clinical Information

Assessment and analysis of likelihood of achieving ongoing pregnancy and live birth also incorporates the use of clinical fertility-associated information, such as phenotypic and/or environmental characteristics. Exemplary clinical information is provided in Table 3 below.

TABLE 3
Clinical information

Cholesterol levels on different days of the menstrual cycle
Age of first menses for patient and female blood relatives (e.g. sisters, mother,
grandmothers)
Age of menopause for female blood relatives (e.g. sisters, mother, grandmothers)
Number of previous pregnancies (biochemical/ectopic/clinical/fetal heart beat detected, live
birth outcomes), age at the time, and outcome for patient and female blood relatives (e.g.
sisters, mother, grandmothers)
Diagnosis of Polycystic Ovarian Syndrome
bAFC
number of embryos transferred
PGS
Female Hormone levels, such as AMH, LSH, FSH and E2
History of hydrosalpinx or tubal occlusion
History of endometriosis, pelvic pain, or painful periods
Cancer history/type of cancer/treatment/outcome for patient and female blood relatives
(e.g. sisters, mother, grandmothers)
Age that sexual activity began, current level of sexual activity
Smoking history for patient and blood relatives
Travel schedule/number of flying hours a year/time difference changes of more than 3 hours
(Jetlag and Flight-associated Radiation Exposure)
Nature of periods (length of menses, length of cycle)
Biological age (number of years since first menses)
Birth control use
Drug use (illegal or legal)
Body mass index (current, lowest ever, highest ever)
History of polyps
History of hormonal imbalance
History of amenorrhoea
History of eating disorders
Alcohol consumption by patient or blood relatives
Details of mother's pregnancy with patient (i.e. measures of uterine environment):
any drugs taken, smoking, alcohol, stress levels, exposure to plastics (i.e. Tupperware),
composition of diet (see below)
Sleep patterns: number of hours a night, continuous/overall
Diet: meat, organic produce, vegetables, vitamin or other supplement consumption,
dairy (full fat or reduced fat), coffee/tea consumption, folic acid, sugar (complex,
artificial, simple), processed food versus home cooked.
Exposure to plastics: microwave in plastic, cook with plastic, store food in plastic,
plastic water or coffee mugs.
Water consumption: amount per day, format: straight from the tap, bottled water (plastic or
bottle), filtered (type: e.g. Britta/Pur)
Residence history starting with mother's pregnancy: location/duration
Environmental exposure to potential toxins for different regions (extracted from government
monitoring databases)
Health metrics: autoimmune disease, chronic illness/condition
Pelvic surgery history
Life time number of pelvic X-rays
History of sexually transmitted infections: type/treatment/outcome
Female reproductive hormone levels: follicle stimulating hormone, anti-Müllerian
hormone, estrogen, progesterone
Stress
Thickness and type of endometrium throughout the menstrual cycle.
Age
Height
Fertility treatment history and details: history of hormone stimulation, brand of drugs used,
basal antral follicle count, follicle count after stimulation with different protocols,
number/quality/stage of retrieved oocytes/development profile of embryos resulting from in
vitro insemination (natural or ICSI), details of IVF procedure (which clinic,
doctor/embryologist at clinic, assisted hatching, fresh or thawed oocytes/embryos, embryo
transfer (blood on the catheter/squirt detection and direction on ultrasound), number of
successful and unsuccessful IVF attempts
Morning sickness during pregnancy
Breast size before/during/after pregnancy
History of ovarian cysts
Twin or sibling from multiple birth (mono-zygotic or di-zygotic)
Semen analysis (count, motility, morphology)
Vasectomy
Testosterone levels
Date of last use and/or frequency of use of a hot tub or sauna
Blood type
DES exposure in utero
Past and current exercise/athletic history
Levels of phthalates, including metabolites:
MEP—monoethyl phthalate, MECPP—mono(2-ethyl-5-carboxypentyl) phthalate,
MEHHP—mono(2-ethyl-5-hydroxyhexyl) phthalate, MEOHP—mono(2-ethyl-5-ox-ohexyl)
phthalate, MBP—monobutyl phthalate, MBzP—monobenzyl phthalate, MEHP—mono(2-
ethylhexyl) phthalate, MiBP—mono-isobutyl phthalate, MCPP—mono(3-carboxypropyl)
phthalate, MCOP—monocarboxyisooctyl phthalate, MCNP—monocarboxyisononyl
phthalate
Familial history of Premature Ovarian Failure/Insufficiency
Autoimmunity history-Antiadrenal antibodies (anti-21-hydroxylase antibodies), antiovarian
antibodies, antithyroid anitibodies (anti-thyroid peroxidase, antithyroglobulin)
Additional female hormone levels: Leutenizing hormone (using immunofluorometric assay),
Δ4-Androstenedione (using radioimmunoassay), Dehydroepiandrosterone (using
radioimmunoassay), and Inhibin B (commercial ELISA)
Number of years trying to conceive
Dioxin and PVC exposure
Hair color
Nevi (moles)
Lead, cadmium, and other heavy metal exposure
For a particular ART cycle: the percentage of eggs that were abnormally fertilized, if
assisted hatching was performed, if anesthesia was used, average number of cells contained
by the embryo at the time of cryopreservation, average degree of expansion for blastocyst
represented as a score, average degree of expansion of a previously frozen embryo
represented as a score, embryo quality metrics including but not limited to degree of cell
fragmentation and visualization of a or organization/number of cells contained in the inner
cell mass (ICM), the fraction of overall embryos that make it to the blastocyst stage of
development, the number of embryos that make it to the blastocyst stage of development,
use of birth control, the brand name of the hormones used in ovulation induction,
hyperstimulation syndrome, reason for cancelation of a treatment cycle, chemical pregnancy
detected, clinical pregnancy detected, count of germinal vesicle containing oocytes upon
retrieval, count of metaphase I stage eggs upon retrieval, count of metaphase II stage eggs
upon retrieval, count of embryos or oocytes arrested in development and the stage of
development or day of development post oocyte retrieval, number of embryos transferred
and date in days post-oocyte retrieval that the embryos were transferred, how many
embryos were cryopreserved and at what stage of development

Information regarding the clinical information, such as the information listed in Table 3, can be obtained by any means known in the art. In many cases, such information can be obtained from a questionnaire completed by the subject that contains questions regarding certain clinical data. Additional information can be obtained from a questionnaire completed by the subject's partner and blood relatives. The questionnaire includes questions regarding the subject's clinical traits, such as his or her age, smoking habits, or frequency of alcohol consumption. Information can also be obtained from the medical history of the subject, as well as the medical history of blood relatives and other family members. Additional information can be obtained from the medical history and family medical history of the subject's partner. Medical history information can be obtained through analysis of electronic medical records, paper medical records, a series of questions about medical history included in the questionnaire, and a combination thereof.

In other embodiments, an assay specific to a phenotypic trait or an environmental exposure of interest is used. Such assays are known to those of skill in the art, and may be used with methods of the invention. For example, the hormones may be detected from a urine or blood test. Venners et al. (Hum. Reprod. 21(9): 2272-2280, 2006) reports assays for detecting estrogen and progesterone in urine and blood samples. Venner also reports assays for detecting the chemicals used in fertility treatments.

Similarly, illicit drug use may be detected from a tissue or body fluid, such as hair, urine, sweat, or blood, and there are numerous commercially available assays (LabCorp) for conducting such tests. Standard drug tests look for ten different classes of drugs, and the test is commercially known as a “10-panel urine screen”. The 10-panel urine screen consists of the following: 1. Amphetamines (including Methamphetamine) 2. Barbiturates 3. Benzodiazepines 4. Cannabinoids (THC) 5. Cocaine 6. Methadone 7. Methaqualone 8. Opiates (Codeine, Morphine, Heroin, Oxycodone, Vicodin, etc.) 9. Phencyclidine (PCP) 10. Propoxyphene. Use of alcohol can also be detected by such tests.

Numerous assays can be used to tests a patient's exposure to plastics (e.g., Bisphenol A (BPA)). BPA is most commonly found as a component of polycarbonates (about 74% of total BPA produced) and in the production of epoxy resins (about 20%). As well as being found in a myriad of products including plastic food and beverage contains (including baby and water bottles), BPA is also commonly found in various household appliances, electronics, sports safety equipment, adhesives, cash register receipts, medical devices, eyeglass lenses, water supply pipes, and many other products. Assays for testing blood, sweat, or urine for presence of BPA are described, for example, in Genuis et al. (Journal of Environmental and Public Health, Volume 2012, Article ID 185731, 10 pages, 2012).

### Methodologies for Assessing Likelihood of Achieving Pregnancy/Live Birth

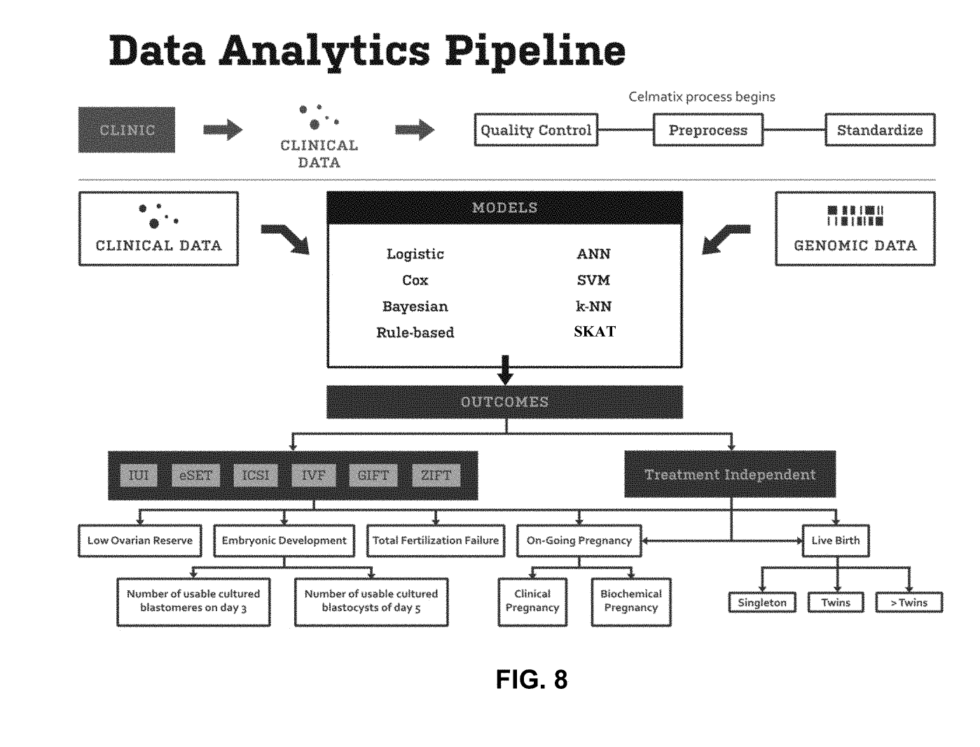

The present invention provides methods for generating a likelihood of achieving ongoing pregnancy in an individual by combining both clinical and genetic data. A general overview of a data analytic pipeline for implementing these methods is provided in FIG. 8. Methods for generating a likelihood of achieving ongoing pregnancy generally involve the determination of one or more correlations between clinical characteristics and known pregnancy and infertility-related outcomes from a reference set of data to provide a model representative of a cumulative probability of ongoing pregnancy over “N” IVF cycles. The methods further involve the determination of one or more correlations between genetic characteristics and known pregnancy and infertility-related outcomes from the reference set of data to adjust the model. The model can then be applied to the input data to generate the likelihood of achieving ongoing pregnancy in the subject.

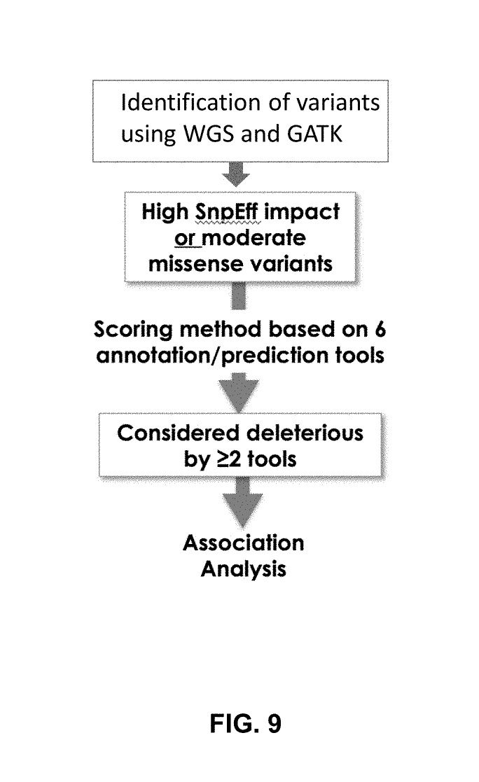

FIG. 9 illustrates a method for determining the impact of genetic characteristics on the cumulative probability of achieving ongoing pregnancy. First, variants within genes and genetic regions, including those described above, are identified. In a preferred embodiment, whole genome sequencing is conducted on DNA extracted from whole blood samples using the Illumina HiSeq platform. As described above, variants can be called using standard Genome Analysis Toolkit (GATK) methods.

Once the variants are called, a customized pipeline is used to identify deleterious variants among the genetic signatures of patients. Deleterious variants can be determined using, for example, the SnpEff and Variant Effect Predictor (www.ensembl.org) engines. SnpEff is capable of rapidly categorizing the effects of SNPs and other variants in whole genome sequences. See, Cingolani et al., A program for annotating and predicting the effects of single nucleotide polymorphisms, SnpEff: SNPs in the genome of Drosophila melanogaster strain w¹¹¹⁸; iso-2; iso-3; Landes Bioscience, 6:2, 1-13; April/May/June 2012, incorporated herein by reference. Variants predicted to have a high impact or be ‘moderate missense variants’ (moderate is defined by SnpEff as causing an amino acid change) using programs such as SnpEff are then selected.

Upon identification of these high and moderate impact variants, the variants are then passed through a scoring system based on various annotation tools. One of ordinary skill in the art would understand that both molecular and computational approaches are available for annotating variants (e.g. by comparing to a known database, through the use of ANOVA technology, through the use of multivariant analysis). Exemplary annotation tools include the Database for Annotation, Visualization and Integrated Discover (DAVID). Nature Protocols 2009; 4(1):44; and Nucleic Acids Res. 2009; 37(1):1, incorporated herein by reference.

Variants that were considered deleterious by at least two annotation tools were then passed through to an association analysis to determine whether the genetic variant signatures obtained from the subjects are associated with their cumulative odds of ongoing pregnancy.

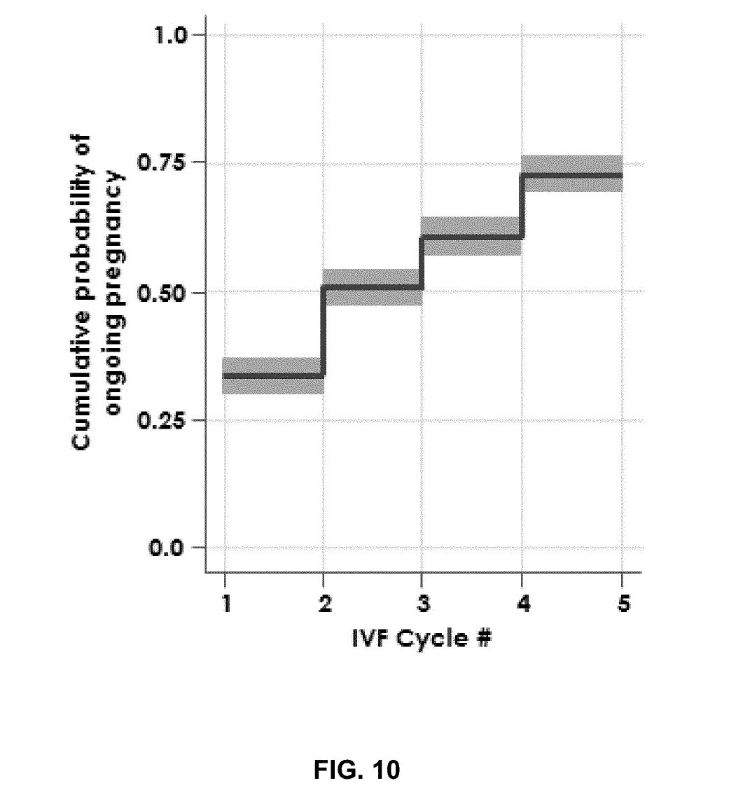

The association analysis involves the use of any one of a number of models to calculate cumulative odds of ongoing pregnancy for a group of subjects, such as a cohort of patients, over a number N of IVF cycles, as shown in FIG. 10. The model incorporates and adjusts for clinical information, such as the phenotypical and environmental characteristics listed in Table 3, obtained from the group of subjects. For example, the model can be adjusted for the subjects' age, bAFC, AMH, number of embryos transferred, PGS, day 3 LSH, day 3 FSH, day 3 E2, etc.

Suitable methods include, without limitation, logistic regression, ordinal logistic regression, linear or quadratic discriminant analysis, clustering, principal component analysis, nearest neighbor classifier analysis, and proportional hazards models.

Logistic regression analysis may be used to generate an odds ratio and relative risk for each characteristic. Method of logistic regression are described, for example in, Ruczinski (Journal of Computational and Graphical Statistics 12:475-512, 2003); Agresti (An Introduction to Categorical Data Analysis, John Wiley & Sons, Inc., 1996, New York, Chapter 8); and Yeatman et al. (U.S. patent application number 2006/0195269), the content of each of which is hereby incorporated by reference in its entirety.

Some embodiments of the present invention provide generalizations of the logistic regression model that handle multicategory (polychotomous) responses. Such embodiments can be used to discriminate an organism into one or more prognosis groups (e.g., good prognosis, poor prognosis). Such regression models use multicategory logit models that simultaneously refer to all pairs of categories, and describe the odds of response in one category instead of another. Once the model specifies logits for a certain (J-1) pairs of categories, the rest are redundant. See, for example, Agresti, An Introduction to Categorical Data Analysis, John Wiley & Sons, Inc., 1996, New York, Chapter 8, which is hereby incorporated by reference.

Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. In one embodiment, the selected fertility-associated phenotypic traits serve as the requisite continuous independent variables. The prognosis group classification of each of the members of the training population serves as the dichotomous categorical dependent variable. For more information on linear discriminant analysis, see Duda, Pattern Classification, Second Edition, 2001, John Wiley & Sons, Inc; and Hastie, 2001, The Elements of Statistical Learning, Springer, New York; Venables & Ripley, 1997, Modern Applied Statistics with s-plus, Springer, New York, incorporated herein by reference.

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

In some embodiments of the present invention, decision trees are used to classify patients using expression data for a selected set of molecular markers of the invention. Decision tree algorithms belong to the class of supervised learning algorithms. The aim of a decision tree is to induce a classifier (a tree) from real-world example data. This tree can be used to classify unseen examples which have not been used to derive the decision tree. In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, but are not limited to classification and regression trees (CART), multivariate decision trees, ID3, and C4.5.

In some embodiments, the fertility-associated characteristics are used to cluster a training set. Additional information and examples are described in Duda and Hart, Pattern Classification and Scene Analysis, 1973, John Wiley & Sons, Inc., New York; Kaufman and Rousseeuw, 1990, Finding Groups in Data: An Introduction to Cluster Analysis, Wiley, New York, N.Y.; Duda, Pattern Classification, Second Edition, 2001, John Wiley & Sons, Inc; and Hastie, 2001, The Elements of Statistical Learning, Springer, New York; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Other algorithms for analyzing associations are known. For example, the stochastic gradient boosting is used to generate multiple additive regression tree (MART) models to predict a range of outcome probabilities. A different approach called the generalized linear model, expresses the outcome as a weighted sum of functions of the predictor variables. The weights are calculated based on least squares or Bayesian methods to minimize the prediction error on the training set. A predictor's weight reveals the effect of changing that predictor, while holding the others constant, on the outcome. In cases where one or more predictors are highly correlated, in a phenomenon known as collinearity, the relative values of their weights are less meaningful; steps must be taken to remove that collinearity, such as by excluding the nearly redundant variables from the model. Thus, when properly interpreted, the weights express the relative importance of the predictors. Less general formulations of the generalized linear model include linear regression, multiple regression, and multifactor logistic regression models, and are highly used in the medical community as clinical predictors.

In a preferred embodiment, a proportional hazards model, such as the Cox proportional hazards model, is used to determine the cumulative probability of ongoing pregnancy in a group of subjects, as shown in FIG. 10. See e.g., Cox, David R (1972). “Regression Models and Life-Tables”. Journal of the Royal Statistical Society, Series B. 34 (2): 187-220, incorporated herein by reference. Proportional hazards models relate the time that passes before some event occurs to one or more covariates that may be associated with that quantity of time, wherein the unique effect of a unit increase in a covariate is multiplicative with respect to the hazard rate (e.g., odds of achieving ongoing pregnancy/live birth).

To further enhance the predictive power of the analysis, genetic information from the subjects can also be incorporated. One method for determining the effect that genetic information has on the cumulative odds of ongoing pregnancy includes the sequence kernel association testing (SKAT) method. See Wu M C, Lee S, Cai T, Li Y, Boehnke M, Lin X. Rare-Variant Association Testing for Sequencing Data with the Sequence Kernel Association Test. American Journal of Human Genetics. 2011; 89(1):82-93. doi:10.1016/j.ajhg.2011.05.029, incorporated herein by reference.

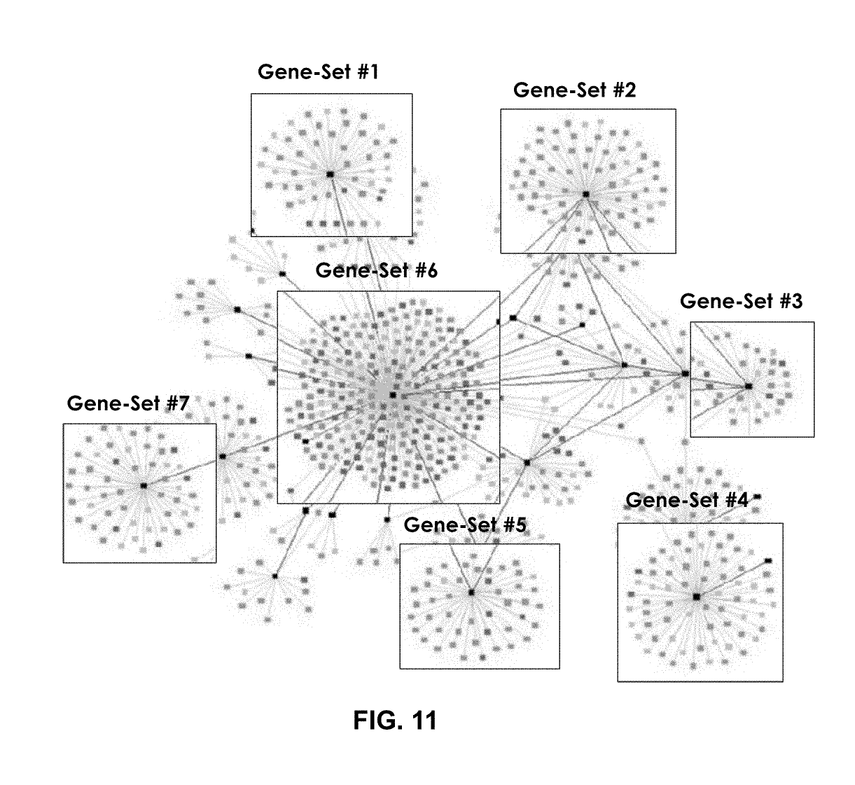

SKAT is a single nucleotide polymorphism set (SNP-set) or gene set level methodology for testing if SNP-sets are associated with phenotypes (continuous or discrete) of interest, as shown in FIG. 11. SNP-sets can include genes, functional biological classifications, genomic regions, etc. These sets are required to be defined prior to performing a SKAT analysis. Gene sets can be defined in any number of ways, such as through use of a fertility-centric database, as described in more detail below.

The SKAT method lends an improvement over SNP-level analyses by reducing the burden of correcting for multiple comparisons, thereby increasing the power to detect true associations. SKAT aggregates SNP-level score test statistics within a SNP-set to compute a P-value for SNP-set level significance. Additionally, SKAT allows for the incorporation of covariates, which allows the method to identify if SNP-sets are correlated with phenotypes of interest even after adjusting for other variables.

SKAT makes no assumption as to the direction of the effect of individual variants on the phenotype, and as such, is a powerful approach for detecting SNP-set level associations in cases where individual SNPs within a category may have differential effects on the phenotype of interest. SKAT assumes that the effects of SNPs on the phenotype follow a distribution with a mean of zero (i.e., no effect on the phenotype) and variance σ². SKAT utilizes a variance-components test of the hypothesis that the variance of the SNP effects is non-zero; i.e., σ²≠0, which provides evidence that there is a SNP-set level association.

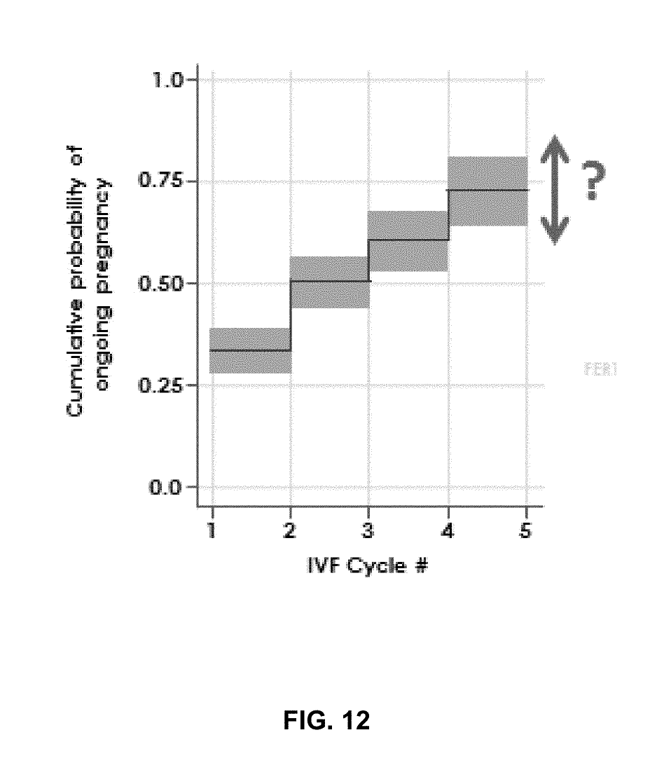

Because SKAT only provides a P-value for the evidence of an association between the SNP-set and the phenotype of interest, but no measure of the magnitude or direction of this effect, as illustrated in FIG. 12, burden testing can be completed to enhance the results of the SKAT analysis.

Burden tests collapse individual variant-level genetic information to the SNP-set level (e.g., gene or functional classification level). For example, each patient can be assigned a genetic burden score within a given functional classification by computing a sum score of the total number of deleterious mutations each patient had within each classification. Burden scores can be treated as continuous or categorized into discrete dichotomous indicators for whether the patient had more than average or less than or equal to average number of mutations within this category relative to the rest of the sample. Burden scores can then be incorporated into standard regression models, which can also control for clinical metrics known to be associated with the phenotype of interest. For example, discrete-time proportional hazards models of the number of IVF treatment cycles until a patient achieves ongoing pregnancy may incorporate genetic burden in addition to known clinical predictors of IVF success. A coefficient from such a model would indicate the effect genetic burden has on achieving ongoing pregnancy during IVF treatment, after controlling for known clinical correlates to IVF success.

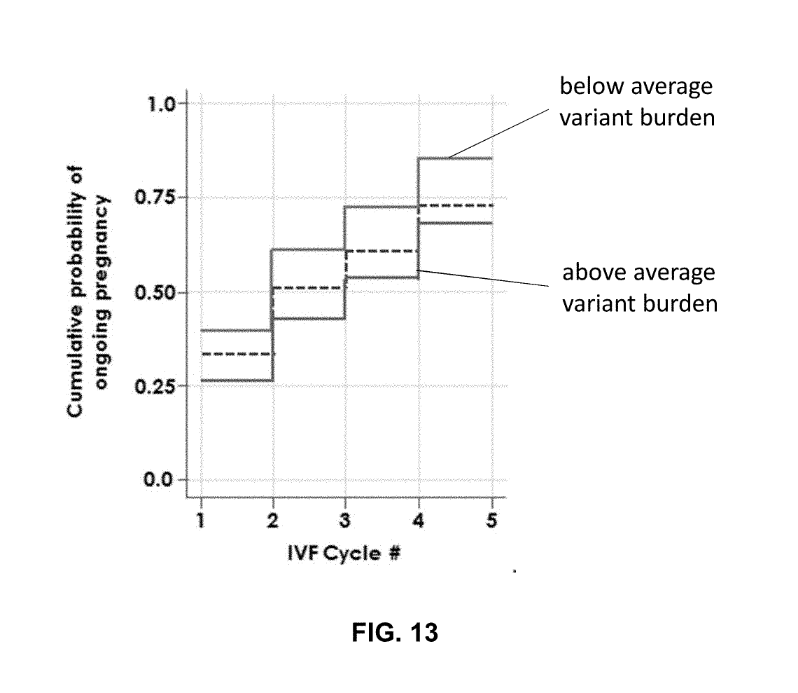

In one embodiment, SKAT is followed with burden testing to elucidate the direction of the effects of genetic information on the odds of achieving ongoing pregnancy, as determined by the Cox proportional hazards method. For example, burden testing is performed by computing a sum score of the total number of deleterious mutations each patient had within each gene category. These scores were then transformed into dichotomous indicators for whether the patient had more than average or less than or equal to average number of mutations within this category relative to the rest of the sample. These indicators were then incorporated into a discrete-time proportional hazards model of the number of IVF treatment cycles until a patient achieved ongoing pregnancy, as shown in FIG. 13.

Accordingly, by adjusting models according to SKAT-analysis results, one is able to see whether there is statistical evidence that genomic information, at the category level (e.g. functional biological classification level), provides additional information beyond known clinical metrics that is sufficient to significantly affect the model, and therefore be associated with the odds of achieving ongoing pregnancy.

### Systems

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the female subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTMLS, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

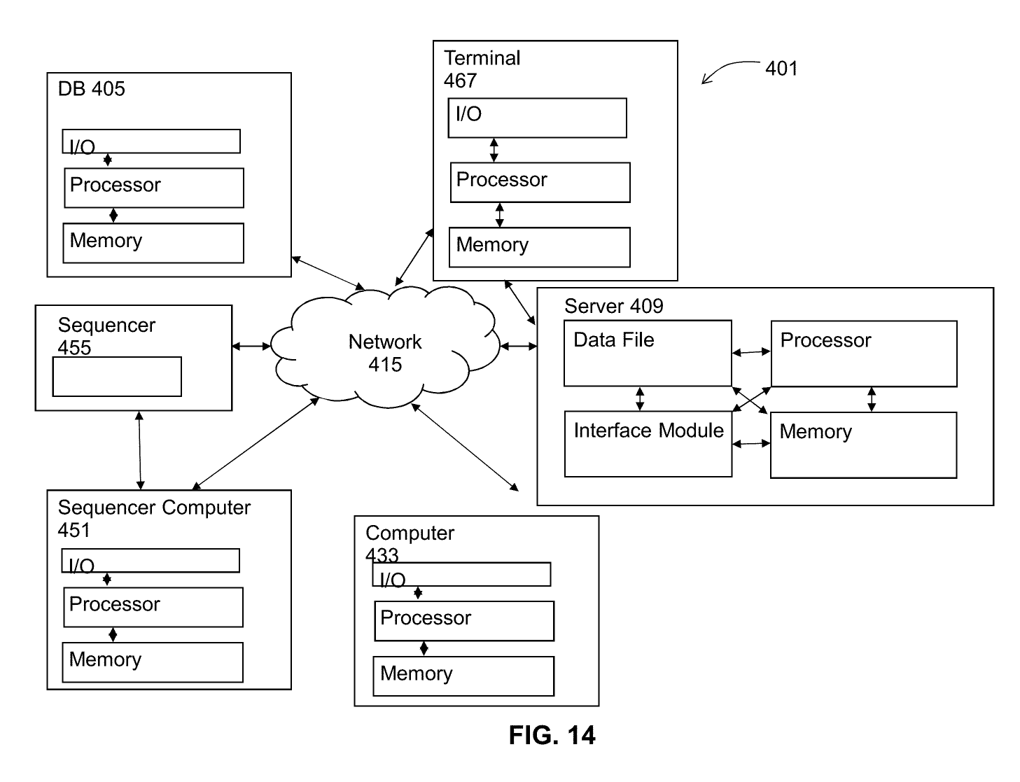

In an exemplary embodiment shown in FIG. 14, system 401 can include a computer 433 (e.g., laptop, desktop, or tablet). The computer 433 may be configured to communicate across a network 415. Computer 433 includes one or more processor and memory as well as an input/output mechanism. Where methods of the invention employ a client/server architecture, any steps of methods of the invention may be performed using server 409, which includes one or more of processor and memory, capable of obtaining data, instructions, etc., or providing results via interface module or providing results as a file. Server 409 may be engaged over network 415 through computer 433 or terminal 467, or server 415 may be directly connected to terminal 467, including one or more processor and memory, as well as input/output mechanism. In some embodiments, systems include an instrument 455 for obtaining sequencing data, which may be coupled to a sequencer computer 451 for initial processing of sequence reads

Memory according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

Other embodiments are within the scope and spirit of the invention. For example, due to the nature of software, functions described above can be implemented using software, hardware, firmware, hardwiring, or combinations of any of these. Features implementing functions can also be physically located at various positions, including being distributed such that portions of functions are implemented at different physical locations.

### Example 1

In this example, proprietary bioinformatics pipelines and statistical analysis were used to identify subclinical genetic factors affecting the ability to achieve live birth.

### Study Design and Methodology

Study subjects: The study subjects consisted of 227 women undergoing IVF treatment at four fertility clinics in the US between 2012 and 2015.

Whole blood samples were taken from each of the study subjects. Genomic DNA was extracted from the whole blood. Whole genome sequences (with an average read depth of 30×) were generated using Illumina HiSeq platform. The sequences generated were then analyzed using GATK standard methods to call variants. Variants, such as single nucleotide polymorphism (SNPs) predicted to disrupt gene function were identified using SNPeff, a variant effect prediction tool. Those variants that had either a high impact predicted by SNPeff, or that were ‘moderate missense variants’ (defined by SNPeff as causing an amino acid change) were then passed through a scoring system based on six different variant annotation tools. Those variants considered deleterious by at least two of these tools were then passed to an association analysis.

Statistical Analysis:

The likelihood of live birth (LB) was calculated with a Cox proportional hazards model using restrospective data from greater than 80,000 IVF treatment cycles across 12 clinics in the US. This model was used to stratify patients into four groups based on prognosis and outcome:

1) Good prognosis (GP, upper quartile) and shorter time to LB (1 cycle to LB): GP-SO
2) Good prognosis and longer time to LB (>1 cycles to or no LB): GP-LO
3) Poor prognosis (PP, lower quartile) and shorter time to LB (≤2 cycles to LB): PP-SO
4) Poor prognosis and longer time to LB (>2 cycles to LB): PP-LO.

### Results

Significant differences in mean age between the patients in the GP and PP groups were revealed: 29.7 vs. 35.8, respectively (p<0.001). The majority of the patients in the PP group were diagnosed with DOR (˜57%) while the majority of the patients in the GP group were idiopathic (˜49%). There were no statistically significant differences in age or BMI between the GP-SO and the GP-LO groups. Of over 25 different biological classifications relating to reproductive function, oogenesis was the only classification whose disruption was significantly assocated with longer time to or lack of LB in both GP and PP patients.

### Conclusions:

This study suggests that subclinical, genetic markers of oocyte quality may hold diagnostic value independent of phenotypic biomarkers of fertility potential such as age and hormone levels. This information can bring clarity to currently unexplained cases of infertility and bring greater efficiency to infertility care and treatment.

### Example 2

In this example, proprietary bioinformatics pipelines and statistical analysis were used to identify subclinical genetic factors affecting the ability to achieve ongoing pregnancy.

### Study Design and Methodology

Study Subjects:

The study subjects consisted of 261 women undergoing IVF treatment at four fertility clinics in the US between 2012 and 2016. Key metrics for the cohort are as follows:

| | |
|---|---|
| Age | 33.3 |
| bAFC | 14.22 |
| Day 3 FSH | 7.41 |
| Avg # IVF Cycles | 1.85 |
| Ongoing Pregnancy Rate | 54% |

DNA Sequencing Analysis:

Whole blood samples were taken from each of the study subjects. Genomic DNA was extracted from the whole blood. Whole genome sequences (with an average read depth of 30×) were generated using Illumina HiSeq platform. The sequences generated were then analyzed using GATK standard methods to call variants. Variants, such as single nucleotide polymorphism (SNPs) predicted to disrupt gene function were identified using SNPeff, a variant effect prediction tool. Those variants that had either a high impact predicted by SNPeff, or that were ‘moderate missense variants’ (defined by SNPeff as causing an amino acid change) were then passed through a scoring system based on six different variant annotation tools. Those variants considered deleterious by at least two of these tools were then passed to an association analysis.

Statistical Analysis:

Sequence kernel association testing (SKAT) was used to test the hypotheses that specific sets of variants were correlated with the odds of achieving ongoing pregnancy after controlling for clinical metrics. Specifically, SKAT was utilized in a discrete-time proportional hazards modelling framework of the number of in-vitro fertilization (IVF) treatment cycles until a patient achieves ongoing pregnancy.

Burden testing was performed by computing a sum score of the total number of deleterious mutations each patient had within each gene category. These scores were then transformed into dichotomous indicators for whether the patient had more than average or less than or equal to average number of mutations within this category relative to the rest of the sample. These indicators were then incorporated into a discrete-time proportional hazards model of the number of IVF treatment cycles until a patient achieved ongoing pregnancy, as shown in FIG. 8.

SKAT and burden testing models controlled for known clinical correlates to IVF treatment success, including age, basal antral follicle count (bAFC), anti-Mullerian hormone (AMH), the number of embryos transferred, preimplantation genetic screening (PGS), and day three levels of luteinizing hormone (day 3 LH), follicle-stimulating hormone (day 3 FSH), and estradiol (day 3 E2). Results of the models indicate whether or not there is statistical evidence that genomic information, at the gene category level, provides additional information beyond known clinical metrics about the odds of achieving ongoing pregnancy in IVF treatment.

### Results

The results of the SKAT analysis are presented in Table 4. Listed P-values indicate the significance level for the association between the variant category and the odds of achieving ongoing pregnancy in IVF. The models were adjusted for patient age, PGS, bAFC, AMH, number of embryos transferred, day 3 LH, FSH, and E2. There was a significant association between genetic variants in the oogenesis classification and the odds of achieving ongoing pregnancy after controlling for known clinical metrics (P=0.020). Folliculogenesis, post-implantation development, and neuroendocrine axis were associated with the odds of ongoing pregnancy at a trend level.

TABLE 4
Sequence kernel association
testing (SKAT) results.

| Category | P-Value |
|---|---|
| Oogenesis | 0.020* |
| Folliculogenesis | 0.051 |
| Post-implantation development | 0.073 |
| Neuroendocrine axis | 0.091 |
| Gonadogenesis | 0.107 |
| Placentation (embryonic) | 0.156 |
| Placentation (uterine) | 0.273 |
| Oocyte-embryo transition | 0.276 |

The adjusted odds ratio (aOR) for the odds of achieving ongoing pregnancy between patients with more than average number of deleterious variants in a gene category, relative to patients with less than or equal to average number of deleterious variants is presented in Table 5 below. Results of this model indicated that patients who had more than average number of mutations within the oogenesis classification had 0.48 times the odds of achieving ongoing pregnancy on a given cycle, relative to a patient with less than or equal to the average number of mutations (aOR=0.48, 95% CI [0.27, 0.86], P=0.014). No other gene categories reached statistical significance.

TABLE 5
Adjusted odds ratio for odds of achieving ongoing pregnancy

| Category | aOR | 95% CI | P-value |
|---|---|---|---|
| Oogenesis | 0.48 | [0.27, 0.86] | 0.014 |
| Oocyte-embryo transition | 1.71 | [1.00, 2.96] | 0.052 |
| Folliculogenesis | 1.40 | [0.87, 2.24] | 0.163 |
| Placentation (uterine) | 0.78 | [0.51, 1.20] | 0.258 |
| Post-implantation development | 1.24 | [0.76, 2.01] | 9.388 |
| Gonadogenesis | 1.22 | [0.73, 2.03] | 0.452 |
| Placentation (embryonic) | 1.03 | [0.63, 1.69] | 0.901 |
| Neuroendocrine axis | 1.00 | [0.64, 1.55] | 0.998 |

### Conclusions:

Similar to Example 1, this study suggests that subclinical, genetic markers of oocyte quality may hold diagnostic value independent of phenotypic biomarkers of fertility potential such as age and hormone levels. This information can bring clarity to currently unexplained cases of infertility and bring greater efficiency to infertility care and treatment.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for generating a likelihood of achieving ongoing pregnancy in a subject, the method comprising
obtaining reference data representative of one or more clinical characteristics and one or more genetic characteristics from a reference set of subjects;
obtaining input data representative of one or more clinical characteristics and one or more genetic characteristics from a subject;
using a computer system comprising a processor coupled to memory and having executable code for:
training the reference data by determining one or more correlations between the one or more clinical characteristics from the reference data and known pregnancy and infertility-related outcomes to provide a model representing a cumulative probability of ongoing pregnancy;
further training the reference by determining one or more correlations between the one or more genetic characteristics from the reference data and known pregnancy and infertility-related outcomes to adjusting the model; and
applying the model to the input data to generate the likelihood of achieving ongoing pregnancy in the subject.

2. The method of claim 1, wherein the one or more genetic characteristics comprising genetic variations.

3. The method of claim 2, wherein the genetic variations comprise mutations from one or more genes within fertility-related biological classifications selected from the group consisting of: oogenesis, folliculogenesis, post-implantation development, neuroendocrine axis, gonadogenesis, embryonic placentation, uterine placentation, and oocyte-embryo transition.

4. The method of claim 3, wherein at least one of the fertility-related biological classifications comprises oogenesis.

5. The method of claim 4, wherein the genes comprise one or more selected from the group consisting of: ACTL6A, AHR, ATM, ATR, AURKA, AURKB, BARD1, BAX, BHMT, BMP15, BMP4, BMP7, BNC1, BRCA1, BRCA2, BUB1, CDK1B, CTCF, DAZL, DDX20, DIAPH2, EEF1A1, EIF2B2, EIF2B5, ESR2, FMN2, FMR1, FOXL2, FOXO3, GDF9, HSF1, IL6ST, KDM1B, KHDC1, KHDC3L, LHCGR, LIFR, MAD1L1, MAD2L1, MCM8, MTA2, MTOR, MTRR, MYC, NLRP11, NLRP13, NLRP14, NLRP4, NLRP5, NLRP7, NLRP8, NLRP9, NOBOX, NOG, NPM2, NTF4, OAS1, OOEP, PLA2G4C, PMS2, POLG, PRDM1, PRLR, RFPL4A, SCARB1 TACC3, TAF4B, TLE6, TP63, TP73 TSC2, ZFX, ZP1, ZP2, ZP3, and ZP4.

6. The method of claim 3, wherein at least one of the fertility-related biological classifications comprises folliculogenesis.

7. The method of claim 6, wherein the genes comprise one or more selected from the group consisting of: ACVR1, ACVR1C, ACVR1C, AHR, AR, BAX, BMP15, BMP4, BMP7, CDKN1B, CENPI, DDX20, EEF1A1, EIF2B2, EIF2B5, ESR1, ESR2, FRM1, FOXE1, FOXL2, FOXO3, FSHR, FST, GALT, GDF3, GDF9, IGF1, IL6ST, INHA, KLF4, LHB, LCGR, MCM8, MTOR, MYC, NOBOX, NOG, NTF4, OAS1, PRLR, PROKR1, PROKR2, TAF4B, TGFB1, TP73, TSC2, USP9X, WT1, XPNPEP2, ZFX, ZP2, and ZP3.

8. The method of claim 2, wherein the one or more genetic characteristics further comprise gene products of genes having genetic mutations.

9. The method of claim 1, wherein the obtaining input data comprises:
sequencing nucleic acid from a sample from the subject to produce sequence reads;
comparing the sequence reads to a reference; and
identifying variations in the sequence reads relative to the reference.

10. The method of claim 1, wherein the one or more clinical characteristics is selected from Table 3.

11. The method of claim 1, wherein the training the reference data using the model to determine one or more correlations between the one or more clinical characteristics from the reference data and known pregnancy and infertility-related outcomes comprises the use of a proportional hazards model.

12. The method of claim 1, wherein the further training the reference data using the model to determine one or more correlations between the one or more genetic characteristics from the reference data and known pregnancy and infertility-related outcomes comprises the use of sequence kernel association testing.

13. A method for treating a patient suspected of having impaired fertility, comprising:
obtaining reference data representative of one or more clinical characteristics and one or more genetic characteristics from a reference set of subjects;
obtaining input data representative of one or more clinical characteristics and one or more genetic characteristics from a subject;
using a computer system comprising a processor coupled to memory and having executable code for:
training the reference data by determining one or more correlations between the one or more clinical characteristics from the reference data and known pregnancy and infertility-related outcomes to provide a model representing a cumulative probability of ongoing pregnancy;
further training the reference by determining one or more correlations between the one or more genetic characteristics from the reference data and known pregnancy and infertility-related outcomes to adjusting the model; and
applying the model to the input data to generate the likelihood of achieving ongoing pregnancy in the subject; and.
providing fertility treatment to the patient based on the generated likelihood of achieving ongoing pregnancy.

14. A method for generating a likelihood of achieving ongoing pregnancy in a subject, the method comprising
obtaining reference data representative of one or more clinical characteristics and one or more genetic characteristics from a reference set of subjects;
obtaining input data representative of one or more clinical characteristics and one or more genetic characteristics from a subject;
using a computer system comprising a processor coupled to memory and having executable code for:
using a model to determine a cumulative probability of ongoing pregnancy based on the one or more clinical characteristics from the reference data;
updating the model to account for the one or more genetic characteristics from the reference set of subjects; and
applying the model to the input data to generate the likelihood of achieving ongoing pregnancy in the subject.
